(19) United States
(12) Patent Application Publication
(10) Pub. No.: US 2011/0286960 A1
(43) Pub. Date: Nov. 24, 2011

(54) CANCER THERAPY BY DOCETAXEL AND GRANULOCYTE COLONY-STIMULATING FACTOR (G-CSF)

(75) Inventors: Oded Vainas, Petach Tikva (IL); Vladimir Vainstein, Jerusalem (IL); Ori Inbar, Ramat Gan (IL); Marina Kleiman, Rishon LeZion (IL); Radel Ben-av, Rehovot (IL); Zvia Agur, Tel Aviv (IL)

(73) Assignee: OPTIMATA LTD., Ramat Gan (IL)

(21) Appl. No.: 13/126,929

(22) PCT Filed: Nov. 2, 2009
(86) PCT No.: PCT/IB2009/007541

§ 371 Date: May 18, 2011

Related U.S. Application Data
(60) Provisional application No. 61/110,572, filed on Nov. 2, 2008.

(51) Int. Cl. A61K 38/19; A61P 37/00; G06G 7/60; A61P 35/00

(52) U.S. Cl. 424/85.1; 703/11

(57) ABSTRACT

Neutropenia is the dose-limiting toxicity of the tri-weekly docetaxel (Taxotere®) schedule. Here, we evaluate in Metastatic Breast Cancer (MBC) patients (N=38) a computerized method for predicting docetaxel-induced neutropenia, and use the model to identify improved docetaxel and Granulocyte Colony Stimulating Factor (G-CSF) regimens. Pharmacokinetics/pharmacodynamics (PK/PD) models were created and simulated concomitantly with a mathematical granulopoiesis model. Individual baseline neutrophil counts and docetaxel schedules served as inputs. Our trial validated the model accuracy in predicting nadir timings (r=0.99), grade 3/4 neutropenia (86% success) and neutrophil profiles (r=0.62). Model was robust to CYP3A-induced variability, except for slightly less accurate grade 3/4 neutropenia predictions. Simulations confirm smaller toxicity of the weekly docetaxel regimen than the tri-weekly one, and suggest an optimal G-CSF support for alleviating neutropenia, 60 μg/day QD×3, 6-7 days post-docetaxel, administered tri- and bi-weekly, and 4 days post weekly docetaxel>33 mg/m².

### RELATED APPLICATIONS

This application claims the benefit of U.S. Provisional Application No. 61/110,572 filed on Nov. 2, 2008, which is incorporated herein by reference in its entirety. The disclosure of U.S. application Ser. No. 10/662,345, filed Sep. 16, 2003, is also incorporated herein by reference in its entirety. U.S. Pat. No. 7,266,483 and reference (24) [Vainstein, V., Ginosar, Y., Shoham, M., Ranmar, D. O., Ianovski, A. & Agur, Z., The complex effect of granulocyte colony-stimulating factor on human granulopoiesis analyzed by a new physiologically-based mathematical model, J Theor Biol 234, 311-27 (2005)], are hereby incorporated by reference in their entirety.

### FIELD OF THE INVENTION

The present invention relates generally to construction of bio-mathematical models, and adjusting and validating them according to experimental results. In particular, this invention relates to granulopoiesis and chemotherapy-induced Neutropenia. The calibrated model provides predictions that can be used to identify optimal treatment regimens. The optimal predictions are made to populations of patients or per an individual patient. The invention covers system method that can be used by physicians or drug developers.

### BACKGROUND OF THE INVENTION

The major dose-limiting toxicity for docetaxel is neutropenia (1). Docetaxel is conventionally administered every three weeks, often resulting in grade 3/4 neutropenia (2). Phase II studies of bi-weekly docetaxel schedules in patients of recurrent ovarian cancer, and advanced non-small cell lung cancer (NSCLC), show similar hematological toxicities (3) and antitumor activity, as the tri-weekly docetaxel schedule (4). Several phase II and III studies in breast cancer (5, 6) and NSCLC patients (7-9) show lower incidences of grade 3/4 neutropenia under weekly dosing, while efficacy and progression-free survival are comparable to the tri-weekly schedule.

A common neutropenia alleviating therapy is G-CSF, mainly administered one day post-docetaxel, for 5-6 consecutive days. No grade 4 neutropenia is reported following G-CSF administration post-docetaxel to locally advanced breast cancer patients (10) or advanced NSCLC patients (11). The main goal for the weekly and bi-weekly schedules, with elective G-CSF, is to achieve the highest effective dose per time unit (denoted dose intensity), which maintains admissible neutropenia. However, trial-and-error methodology is still prevailing for determining the dosing schedule and the G-CSF support timing for individual patients, and improved methodology, supported by predictive models, is highly desirable for identifying optimal docetaxel/G-CSF schedules (12). There are hundreds of thousands of different docetaxel/G-CSF schedules that may be considered in order to achieve an optimal regimen. Therefore, trial and error experimentations are not feasible to accomplish this goal.

### BRIEF DESCRIPTION OF THE DRAWINGS

FIG. 1. Schematic description of the combined Docetaxel/granulopoiesis model. Docetaxel (upper box) is represented by a three-compartment PK model, where arrows represent exchange constants of the drug between the central and peripheral compartments (k₁₂, k₂₁, k₁₃, k₃₁), and the elimination rate from the body (k_{cl}). Granulopoiesis (lower box) is described as a pipeline initiated by stem cells inflowing to the myeloblasts compartment, then, sequentially, differentiating into promyelocytes, myelocytes, post-mitotic BM cells, and finally released to the blood as mature neutrophils. G-CSF accelerates proliferation, transition through the mitotic compartment, the release of post mitotic cells to the blood and their apoptosis. Docetaxel affects the mitotic compartments.

FIG. 2. Model predictions compared to clinical outcomes. Model-predicted neutrophil counts over time (solid lines) compared to the observed neutrophil counts (empty circles) of representative MBC patients, treated with different docetaxel schedules: (A) 25-35 mg/m² once weekly, (B-D) 100-75 mg/m² tri-weekly. (E) Model predictions of the nadir days at each cycle of patients receiving a tri-weekly docetaxel vs. the observed nadir days (circles; N=66; calculated correlation coefficient is r=0.99). The dashed line represents the identity line.

FIG. 3. Effects of once-weekly docetaxel schedule on neutropenia, as compared to a tri-weekly schedule. Model-predicted neutropenic response under the tri-weekly docetaxel, 100 mg/m² regimen (solid line) and the weekly 33 mg/m² regimen (dashed-dotted line) in one characteristic patient. The total dose intensity is the same in both simulations (33 mg/m²/week). Horizontal dashed line represents the grade 4 neutropenia threshold. The tri-weekly regimen is predicted to result in grade 4 neutropenia, while the weekly schedule is expected to yield a milder response.

FIG. 4. The effect of G-CSF onset day on the neutropenic response. The distribution of G-CSF onset day in two simulation subsets from all the possible G-CSF combinations were compared. The first subset included simulations where the maximal neutropenia grade is 4 and the grade 3/4 neutropenia duration is more than 4 days (N=1,460; noted as “bad responders”; black bars). The second subset included simulations that resulted in a maximal grade 2 neutropenia (N=1,074; noted as “good responders”; grey bars). The good responders were those with G-CSF administration mainly on days 6-7 post-docetaxel. In contrast, the bad responders were administered with G-CSF mainly on days 1-2 post-docetaxel.

FIG. 5. Administration day of G-CSF post-docetaxel as affecting the neutropenic response. Application of G-CSF, 60 μg/day, QD×3, was simulated using the population model, in different days post chemotherapy: (A) Duration of grade 4 neutropenia, and recovery to baseline, as a function of G-CSF administration. G-CSF administered on days 6-7 following a single 75 mg/m² docetaxel dosing (as in the tri-weekly regimen), caused the fastest recovery to baseline of neutrophil counts (dashed line), with no grade 4 neutropenia (solid line). (B) Long term toxicity as affected by G-CSF administration time. G-CSF administration 4 days post-chemotherapy weekly docetaxel regimen, 33 mg/m² for 21 treatment cycles, is predicted to yield lower toxicity than other G-CSF administration times, both in the first cycle and during the whole treatment.

FIG. 6. Granulopoiesis as a function of the docetaxel/G-CSF regimen. Treatment of MBC patients by G-CSF, 60 μg/day, QD×3, following a single administration of 75 mg/m² docetaxel was simulated using the docetaxel/granulopoiesis model. Simulation results show (A) the counts of blood neutrophils over time as affected by the treatment; docetaxel only (dashed-dotted line); G-CSF administration on day one (dotted line) or six (solid line) post docetaxel; upper limit of grade 4 neutropenia appears as a horizontal dashed line. (B-E) Granulopoiesis progenitor normalized counts as affected by: G-CSF application one day (dotted line) or six days (solid line) post chemotherapy. In the early G-CSF treatment, the G-CSF-driven release of mature cells to blood and the resulting depletion of BM reservoirs precede the chemotherapy-caused nadir in blood neutrophils, accentuated due to no compensation from BM reservoirs. In the later G-CSF application the BM has already recovered, and the release to blood of mature cells from the completely full BM reservoirs compensates the damage caused by docetaxel, leading to a milder neutropenic response, and a faster recovery to baseline.

FIG. 7. Increasing dose intensity of docetaxel treatment. Docetaxel administration was simulated bi-weekly (days 0, 14,28) together with 100 (dashed-dotted line), 200 (dotted line), or 250 (solid line) mg/m² with G-CSF dose of 60 μg/day, 6 days post-docetaxel, QD×3, in each administration. Although neutropenia appeared, the recovery to baseline was sufficient in the next dose. Horizontal dashed line—grade 4 neutropenia.

FIG. 8. Evaluating docetaxel PK/PD model. (A) Docetaxel PK model parameters were evaluated by data taken from Zuylen et al., 2000 using a dose of 100 mg/m2 after a 1 hour i.v. (empty circles). The multi-exponential model behavior (solid line) reflect the experimental outcomes, thus verifying mathematical PK model adequacy. (B) Validation of the docetaxel/granulopoiesis PK/PD model predictions by independent data. The model predictions were plotted as a function of the estimated Area Under the Curve (AUC) of docetaxel plasma concentration (solid line), to be compared with clinical data from a phase I and pharmacokinetic clinical trial, in which cancer patients data, receiving docetaxel 5-115 mg/m2 bi-weekly or tri-weekly (rectangles; Extra et al., 1993). It can be seen that model predictions, based our MBC patient population, stand in good fit to experimental data from patients of various solid cancer diseases.

FIG. 9. An example of the PrediTox calculator's functionality and graphical user interface. A snapshot of PrediTox, a web calculator that may provide personal/general predictions with regards to expected neutropenia following chemotherapy with or without supportive therapy. In particular, this version of PrediTox provides predictions of expected neutropenia following docetaxel or combined docetaxel with G-CSF schedules. A G-CSF optimization algorithm (presented in full in the “Detailed description of the invention” section) can also be implemented, adjusting the optimal G-CSF schedule to the docetaxel regimen and patient/population characteristics. PrediTox covers over 650,000 different initial conditions, and the results of the docetaxel/granulopoiesis model predictions under 0, 60, 150, 240, 300, 480 μg/day G-CSF at different onset post-60, 75, 100, 152 and 150 mg/m² tri-weekly docetaxel, or at different onset post-40, 50, 67, 83 and 100 mg/m² bi-weekly docetaxel, ranging from day 1 to 7 post-docetaxel, for 1 to 5 days, or at different onset post-20, 25, 33, 42 and 50 100 mg/m² weekly docetaxel, ranging from day 1 to 4 post-docetaxel, for 1-3 day, assuming a uniform neutrophil baseline distribution in the range of 2,000-10,000 neutrophils/μl (in increments of 150) as an input, over treatment periods of 3, 6, 12, 18 and 36 weeks. The output of PrediTox presents the following characteristics of the expected neutropenia per each run: the worse neutropenia grade the patient is expected to reach throughout the whole treatment (for at least 24 hours), the overall duration in grade 3/4 (in days), the average duration at grade 3/4 (in days) per docetaxel cycle, the mode grade before next docetaxel administration and the median day of nadir. (A) The snapshot of a run when only docetaxel is administered. Please note the expected severe neutropenia. (B) The snapshot of a run when optimal G-CSF schedule is combined to the same docetaxel regime as in (A). Please note the dramatic reduction in the expected neutropenia.

### DETAILED DESCRIPTION OF THE INVENTION

### Definitions and Abbreviations

AUC: Area Under the Curve

BM: bone marrow

CSFs: Colony-stimulating factors: Colony-stimulating factors (CSFs) are secreted glycoproteins which bind to receptor proteins on the surfaces of hemopoietic stem cells and thereby activate intracellular signaling pathways which can cause the cells to proliferate and differentiate into a specific kind of blood cell (usually white blood cells, for red blood cell formation see erythropoietin). They may be synthesized and administered exogenously. However, such molecules can at a latter stage be detected, since they differ slightly from the endogenous ones in e.g. features of posttranslational modification.

CYP3A: Cytochrome P450, family 3, subfamily A, is a human gene. The CYP3A locus includes all the known members of the 3A subfamily of the cytochrome P450 superfamily of genes. These genes encode monooxygenases which catalyze many reactions involved in drug metabolism and synthesis of cholesterol, steroids and other lipids.

DI: dose intensity

Dose intense docetaxel regimens: docetaxel regimens that are higher than what is clinically approved. For example, for tri-weekly administration 125 and 150 mg/m² of docetaxel, for bi-weekly administration 83 and 100 mg/m² of docetaxel and for weekly administration 42 and 50 mg/m² of docetaxel.

DLT: dose limiting toxicity

DOC: Docetaxel, also know as Taxotere®

Dose intensity: highest effective dose per time unit

G-CSF: Granulocyte Colony-Stimulating Factor

Grades of Neutropenia according “NCI Common Toxicity Criteria, v3”,
G0: Neutropenia Grade 0: above 2,000 neutrophils/μl
G1: Neutropenia Grade 1: below 2,000 neutrophils/μl
G2: Neutropenia Grade 2: below 1,500 neutrophils/μl
G3: Neutropenia Grade 3: below 1,000 neutrophils/μl
G4: Neutropenia Grade 4: below 500 neutrophils/μl
G3/4: Neutropenia Grade 3 or Grade 4: below 1,000 neutrophils/μl and below 500 neutrophils/μl
G0/1/2: Neutropenia Grade 0 or Grade 1 or Grade 2: all cases above 1,000 neutrophils/μl

Improved regimen: a regimen of a drug or combination of drugs that result in reduced toxicity and/or increased efficacy. One of the important toxicities resulted from DOC is Neutropenia. The efficacy of DOC is in a direct relation to its dose intensity. An improved regimen, for instance, could provide a lower toxicity with the same dose intensity as compared to a standard regimen. Alternatively, an improve regimen could reach a higher DOC dose intensity and keep the same level of toxicity in comparison to a standard regimen. An improved regimen could also result with enhanced efficacy and reduced toxicity.

MBC: Metastatic Breast Cancer

Neutropenia: is a hematological disorder characterized by an abnormally low number of neutrophils, the most important type of white blood cell, in the blood. Neutrophils usually make up 50-70% of circulating white blood cells and serve as the primary defense against infections by destroying bacteria in the blood. Hence, patients with neutropenia are more susceptible to bacterial infections and, without prompt medical attention, the condition may become life-threatening (neutropenic sepsis). Neutropenia can be acute or chronic depending on the duration of the illness. A patient has chronic neutropenia if the condition lasts for longer than 3 months. It is sometimes used interchangeably with the term leukopenia (“deficit in the number of white blood cells”), as neutrophils are the most abundant leukocytes, but neutropenia is more properly considered a subset of leukopenia as a whole.

NSCLC: non-small cell lung cancer

PD: pharmacodynamics

peg G-CSF: pegylated Granulocyte-Colony Stimulating Factor

PK: pharmacokinetics

PrediTox: a version of the validated docetaxel/granulopoiesis model presented in this application that provides personal and general predictions regarding neutropenia following chemotherapy with or without supportive therapy. A version of PrediTox that is calibrated on docetaxel and G-CSF is currently presented.

Q21D: one administration every 21 days

Q14D: one administration every 14 days

Q7D: one administration every 7 days

STD: Standard Deviation

Types of cancers which are currently treated using Docetaxel are: Breast Cancer, Lung Cancer, Prostate Cancer, Gastric Cancer and Head & Neck Cancer.

### The Bio-Mathematical Model

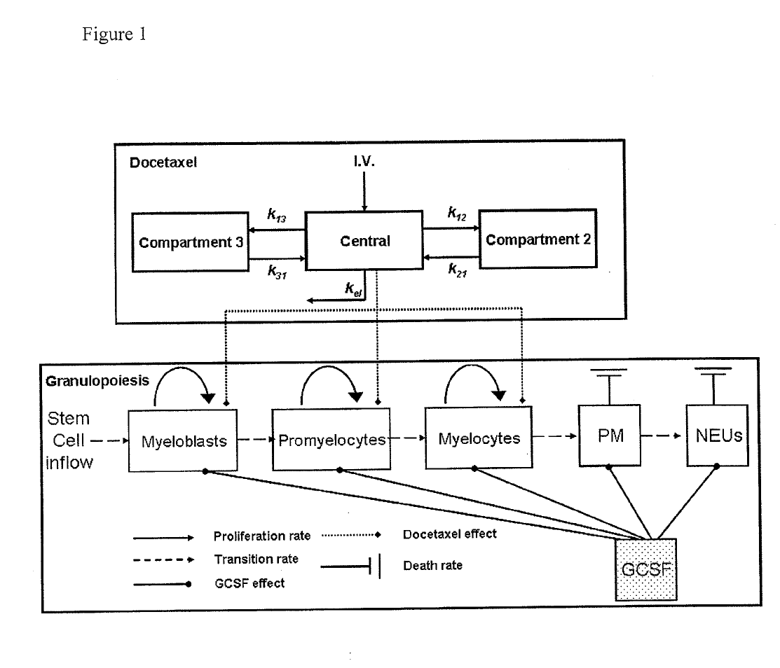

Mathematical models have been previously suggested for studying granulopoiesis in cases of radiation (13, 14), pathologic hematopoiesis (15), bone marrow (BM) transplantation (16), chemotherapy (17) or post-chemotherapy G-CSF support (18-20). Nevertheless, optimal G-CSF supportive protocols have not been studied by any of these models and some of them assume only short-term effects of G-CSF on BM, ignoring important contributions of this agent to safety outcomes (21, 22). To replace the trial-and-error treatment design by scientifically-based decision-making, a granulopoiesis mathematical model was developed accounting for the complex dynamics of mitotic and non-mitotic progenitors, and blood neutrophils with explicit terms of cell-cycle phases (24) and U.S. Pat. No. 7,266,483. The bio-mathematical docetaxel/granulopoiesis model that is used in this application, in general, is presented in U.S. Pat. No. 7,266,483 and reference 24 which are incorporated here by reference in their entirety. G-CSF is modeled in (24) as a feedback molecule governing BM maintenance of steady neutrophils level in blood (23), taking into account G-CSF secretion, diffusion, clearance and interaction with different cell compartments in neutrophil development pipeline, in neutropenic and healthy subjects. In the present work the granulopoiesis model (24) is combined with a mechanism-based pharmacokinetics/pharmacodynamics (PK/PD) modeling methodology presented here (FIG. 1). Using the combined granulopoiesis/drug PK/PD model, the effects of different monotherapy and combination regimens on granulopoiesis can be simulated for identification of improved treatment schedules.

### Bio-Mathematical Model Validation

The granulopoiesis model accounting for neutrophil development in the BM was originally calibrated using literature data (24) and U.S. Pat. No. 7,266,483. The model's prediction accuracy was employed with a conventional method, by which the patients were divided into a “training set”, whose clinical outcomes were used for adjusting model parameters to the given patient population, and a “validation set”, for testing the model-predicted neutrophil profiles of docetaxel-treated patients (25, 26). The individual input data comprised only the patient's baseline neutrophil count and the ascribed docetaxel schedule. All other model parameters (i.e., docetaxel PK, granulopoiesis, G-CSF PK/PD) were constants. Note that our data were combined from Caucasians MBC patients; mainly females see Table 1.

TABLE 1
Patients demographics for MBC study population.

| | |
|---|---|
| Nottingham City | Soroka University |
| Hospital, UK | Hospital, Israel |
| (N = 12), mean | (N = 26), mean |
| (range) | (range) |

| Age | 51 | (36-76) | 55 | (30-80) |
|---|---|---|---|---|
| Gender | | | | |
| Female | 12 | | 25 | |
| Male | 0 | | 1 | |
| Weight (Kg) | 64 | (45-80) | 68 | (45-98) |
| Height (cm) | 162 | (150-170) | 160 | (148-175) |
| Ethnic Group | Caucasians | | Caucasians | |
| Docetaxel Treatment | 152 | (114-274) | 106 | (38-354) |
| Duration (days) | | | | |
| Treatment Regimen | Q21D | | Q21D or Q7D | |
| Dose of docetaxel | 100 | (67.5-100) | 36 | (23-101) |
| (mg/m²) | | | | |

Q21D = docetaxel administered every three weeks; Q7D = docetaxel administered once a week.

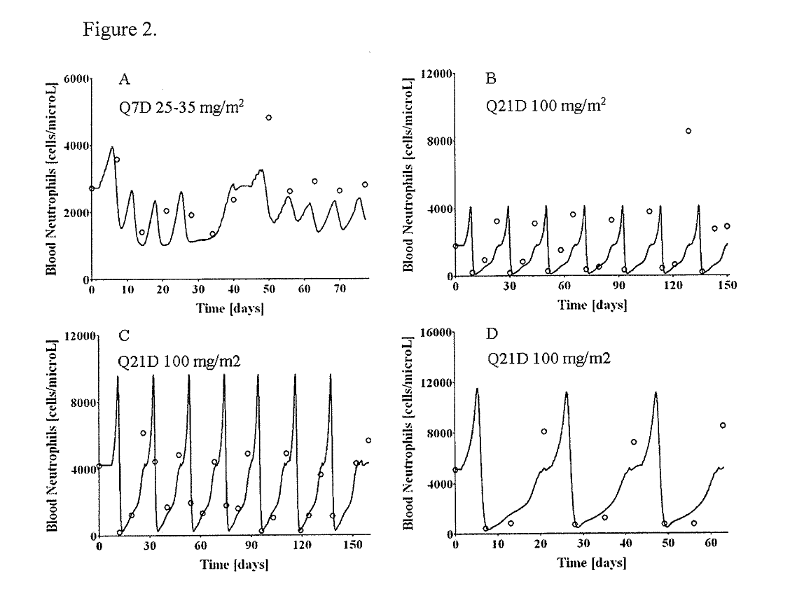

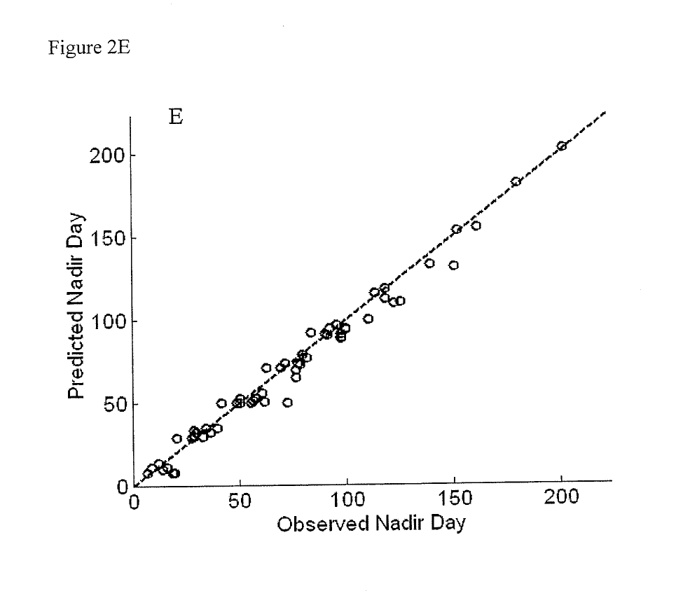

The first and second end-points were defined as achieving high accuracy in predicting the time of nadir for the patients treated with the tri-weekly regimen and the individual neutrophil counts over the treatment period. Nadir is defined as the lowest observable neutrophil count measured at each cycle. Results show a high accuracy in predicting nadir timing at each cycle (r=0.99, P<1.4E-58; FIG. 2E), and a good overall prediction of individual neutrophil profiles (r=0.62, P<5.96E-56), with a mean error in neutrophil counts of ±383 neutrophils/μl (FIG. 2A-D; results for four representative patients).

Our third end-point was defined as achieving high accuracy in predicting grade 3/4 neutropenia (G3/4). Results show positive and negative predictive values of 86% and 83%, respectively (kappa=0.69, P<0.001; positive—grade 3/4 neutropenia, negative—otherwise). Specifically, the model predicted grade 3/4 neutropenia for 12/14 patients who had experienced this toxicity, and grade 2 for two patients.

### Methods Used for the Validation of the Model

Patients. Weekly blood counts were collected from 38 Caucasian MBC patients (Table 1), treated with docetaxel tri-weekly or weekly from two sites (Nottingham City Hospital, Nottingham, UK, and Soroka Medical Center, Be'er Sheva, Israel). Patients from both sites were mixed and randomly divided into a training set, used for adjusting model parameters (N=12), and a validation set (N=26). Docetaxel schedules and neutrophil baselines (median 5,080 neutrophils/μl; range 1,800-15,500) were input for the model. Individual plasma docetaxel measurements were not collected, and prior chemotherapy and performance status were partially recorded, therefore were not included.

### Docetaxel Pharmacokinetic Model

Docetaxel's plasma concentration suggest multi-compartment PK (37, 41). We developed a three-compartment population mean docetaxel PK model for calculating its concentration-time profiles. The central compartment representing blood and two peripheral compartments representing all body tissues that have direct and fast exchange with blood, such as the BM (FIG. 1). In this way, equal or proportional compartmental concentrations can be assumed. Drug distribution was modeled as a linear exchange and elimination process between the connected compartments. Our PK model is mathematically described in Equation 1:

$$\frac{d {X}_{1}}{d t} = {k}_{21} \cdot {X}_{2} + {k}_{31} \cdot {X}_{3} - ( {k}_{21} + {k}_{31} + {k}_{el} ) \cdot {X}_{1} \qquad Eq ( 1 )$$

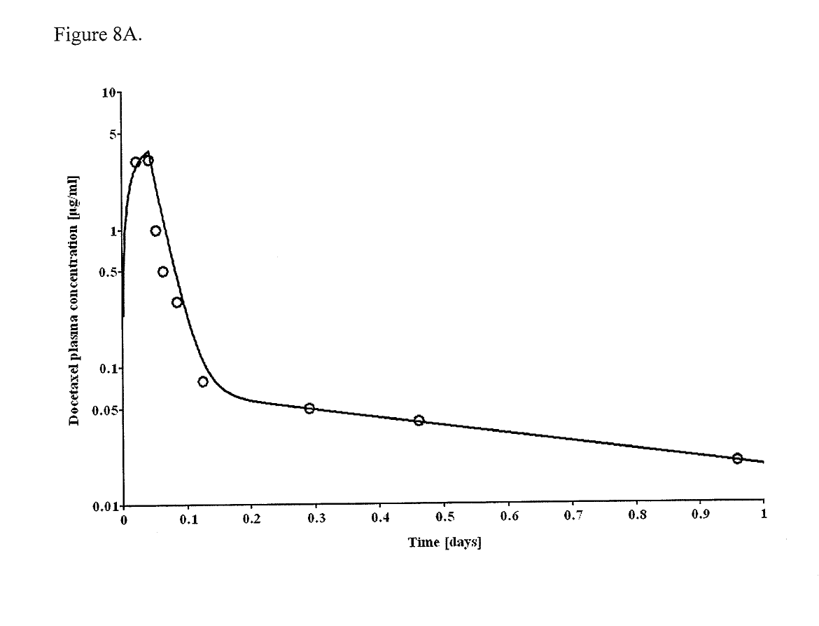

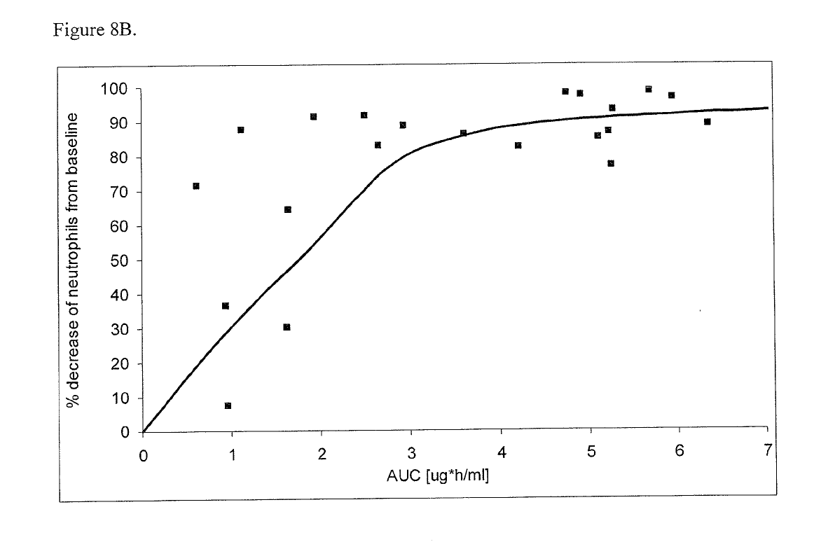

X₁, X₂ and X₃ are the quantities of drug X in the central and the peripheral compartments, respectively. The concentrations are easily calculated using the volumes of the compartments. kₑₗ, and k₁₂, k₂₁, k₃₁ represent the elimination and the kinetic inter-compartment exchange constants, respectively. We assumed constant binding of docetaxel due to lack of individual AAG data. After evaluating the PK model parameters by clinical data (FIG. 8A; ref 30) it was validated by showing good agreement between the model-predicted PK parameters and independent data taken from a PK study of weekly and tri-weekly docetaxel administrations (ref 42; Table 2). The model also predicted docetaxel plasma concentrations of other studies (r=0.738; ref 41, 43-47).

TABLE 2
Docetaxel model predicted vs. experimental PK parameters.

| | 100 mg/m² | | 35 mg/m² | |
|---|---|---|---|---|
| | | Baker et al., | | Baker et al., |
| | Model | 2004; | Model | 2004; |
| Parameter | Predictions | mean ± STD | Predictions | mean ± STD |
| Terminal half life (h) | 12.12 | 17.20 ± 6.20 | 12.12 | 15.60 ± 12.00 |
| AUC (μg*h/ml) | 5.08 | 5.62 ± 2.12 | 1.98 | 1.32 ± 0.42 |
| Cmax (μg/ml) | 3.74 | 4.15 ± 1.35 | 1.78 | 1.85 ± 0.73 |
| Clearance (L/h*m²) | 19.69 | 19.60 ± 5.60 | 19.68 | 29.10 ± 10.20 |

The PK parameters of the model were validated with experimental data from a clinical study, where two groups of patients received 100 and 35 mg/m² separately. In both cases, the predicted PK parameter values reside within the standard deviation of the mean experimental value.
STD = Standard Deviation; AUC = Area Under the Curve

### Pharmacodynamic Modeling of Docetaxel Effects on Granulopoiesis.

Docetaxel effects were modeled as direct killing of neutrophil proliferating progenitors (FIG. 1), which are the most likely targets of docetaxel in granulopoiesis (34, 35). Each effect is related to docetaxel plasma concentration over time (equation 2):

$$E ( C ) = {E}_{max} - \frac{{E}_{max} - {E}_{min}}{1 + {( C / {C}_{nor} )}^{m}} \qquad Eq ( 2 )$$

where E is the measured effect at the given concentration C; Eₘₐₓ and Eₘᵢₙ are the maximal and the minimal possible effects, respectively; Cₙₒᵣ is the drug concentration producing the effect equaling to the average of Eₘₐₓ and Eₘᵢₙ; m is the curve slope at the point [Cₙₒᵣ; E(Cₙₒᵣ)]. The PD parameters were estimated using a cross-entropy algorithm (48) by curve fitting to the training set clinical data. A single set of PD parameters was estimated, best fitting to all the training set data points, when simulated with the docetaxel/granulopoiesis model (noted as a population PD model).

### Methods in the Model Validation.

Baseline neutrophil counts and treatment schedules of each patient were input into the combined docetaxel/granulopoiesis model. The three end-points for model validation were accuracy in predicting nadir days (nadir day is defined as the lowest observable neutrophil count at each cycle), accuracy in predicting grade 3/4 neutropenia (evaluated by Kappa test), and accuracy in predicting neutrophil counts over time (denoted as neutrophils profile) of docetaxel-treated patients. Significance was evaluated using the Pearson correlation test (r) between observed and predicted results, allowing a of ±6 hours time window in nadir prediction evaluation. Note that clinical blood was done once every few days.

### Neutrophil Dynamics Under the Approved Once and Tri-Weekly Docetaxel Schedules

To assess the differences in neutrophil dynamics between the two common MBC docetaxel treatment schedules, 33 mg/m² weekly and 100 mg/m² tri-weekly, (5, 6), these schedules were simulated by our population docetaxel/granulopoiesis model,

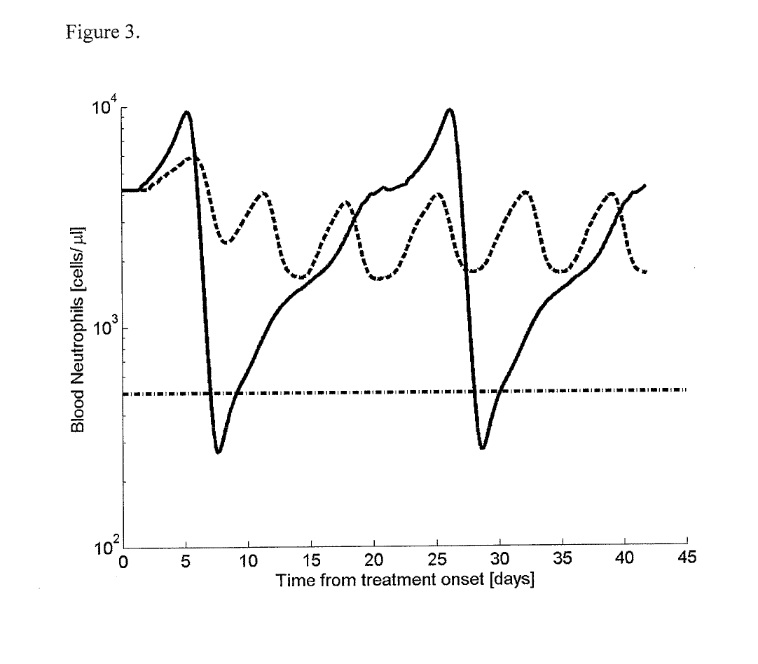

Results suggest that a nadir reaching grade 4, is expected to occur 7-8 days post-docetaxel in patients whose baseline count is ca. 4,200 neutrophils/μl, receiving docetaxel 100 mg/m² tri-weekly, (FIG. 3, solid line). Recovery to baseline in these patients occurs 20 days post-docetaxel (see similarity with clinical results in FIG. 2B-D). These results are supported by docetaxel clinical trial results, where nadir and recovery to baseline were recorded on days 6-10 and 16-22, respectively (17, 28). Our model predicts grade 0/1 neutropenia for the weekly 33 mg/m² docetaxel schedule, with a sufficient neutrophil level recovery (ca. 1,600 neutrophils/μl) to enable subsequent docetaxel administrations (FIG. 3, dashed line).

These results, implicating that fractionation of the total docetaxel dose relieves docetaxel-affected neutropenia, are corroborated by clinical observations (5-9; see also 29).

### Simulations of Various G-CSF Schedules and Determination of Optimal G-CSF.

G-CSF is commonly used as support therapy during docetaxel treatment. However, its optimal timing and dose are yet to be determined. PrediTox covers over 650,000 different initial conditions, and the results of the docetaxel/granulopoiesis model predictions under 0, 60, 150, 240, 300, 480 μg/day G-CSF at different onset post-60, 75, 100, 152 and 150 mg/m² tri-weekly docetaxel, or at different onset post-40, 50, 67, 83 and 100 mg/m² bi-weekly docetaxel, ranging from day 1 to 7 post-docetaxel, for 1 to 5 days, or at different onset post-20, 25, 33, 42 and 50 100 mg/m² weekly docetaxel, ranging from day 1 to 4 post-docetaxel, for 1-3 day, assuming a uniform neutrophil baseline distribution in the range of 2,000-10,000 neutrophils/μl (in increments of 150) as an input, over treatment periods of 3, 6, 12, 18 and 36 weeks.

An optimal G-CSF schedule is selected according the following objectives: minimization of neutropenia grade, neutropenia duration and G-CSF exposure (i.e. the dose and duration of G-CSF). The algorithm also takes into account that the patient's neutrophil level before the next Docetaxel administration should be sufficient for the continuation of the treatment, i.e., grade 0 or 1. Note that neutropenia grade is determined according to the “NCI Common Toxicity Criteria, v3”, e.g., below 500 neutrophils/μl for grade 4, for at least 24 hours.

The G-CSF regimen optimization algorithm calculates the expected neutrophil dynamics for the full spectrum of potential G-CSF regimens in order to find the optimal regime according to an optimization algorithm. An example of such algorithm, the one used in this application, is presented below. The spectrum of G-CSF regimens is given by all possible combinations of G-CSF onset (relative to application of docetaxel), G-CSF dose, and G-CSF duration (176 for combinations for tri and bi weekly or 61 combinations for weekly).

### An Algorithm for Optimization of G-CSF Regimen

Select regimen(s) where the expected neutropenia grade is 0 or 1
If more than one regimen was found then, select the regimen with minimal G-CSF administered

If one was found then, select regimen(s) where the expected neutropenia grade is 2
If more than one regimen was found then, select the ones with mode grade before next docetaxel administration is 0 or 1
   If more than one regimen was found then, select the one with minimal G-CSF administered
If none was found then, select regimen(s) where the expected neutropenia grade is 3 and median grade before next docetaxel administration is 0 or 1
   If more than one regimen was found then, select the ones with the minimal overall duration at grade 3/4
      If more than one regimen was found then, select the ones with the minimal mode grade before next administration
         If more than one regimen was found then, select the ones with the minimal average duration at grade 3/4, per docetaxel cycle
         If more than one regimen was found then, select the one with the minimal G-CSF administered

If none was found then, select regimen(s) where the expected neutropenia grade is 3
If more than one regimen were found then, select ones where the expected neutropenia grade is 3 and median grade before next docetaxel administration is 0 or 1 or 2
   If more than one regimen were found then, select the ones with the minimal overall duration at grade 3/4
      If more than one regimen were found then, select the ones with the minimal mode grade before next docetaxel administration
         If more than one regimen were found then, select the ones with the minimal average duration at grade 3/4, per docetaxel cycle
         If more than one regimen was found then, select the one with the minimal G-CSF administered

If none was found then, select regimen(s) where the expected neutropenia grade is 4
If more than one regimen were found then, select ones where the expected neutropenia grade is 3 and median grade before next docetaxel administration is 0 or 1 or 2
   If more than one regimen were found then, select the ones with the minimal overall duration at grade 3/4
      If more than one regimen were found then, select the ones with the minimal mode grade before next docetaxel administration
         If more than one regimen were found then, select the ones with the minimal average duration at grade 3/4, per docetaxel cycle
         If more than one regimen was found then, select the one with the minimal G-CSF administered

### Timing of G-CSF Support Significantly Affects the Grade and Duration of Docetaxel-Induced Neutropenia

Simulation results suggest that G-CSF application timing is crucial for its efficacy, and that wrong timing may lead to a more severe neutropenia, rather than alleviation of docetaxel-caused toxicity. Analysis of the optimal schedules subset, shows that the selected optimal G-CSF schedules decreased the fraction of the population with grade 3/4 neutropenia in comparison with the no G-CSF application population (28% vs. 100%; p<2.4E-84; Table 3). It is interesting to note that the optimal regimen is also substantially better than the outcome of the collection of numerous G-CSF schedules. Taking the “fraction of the population with grade 3/4 neutropenia” over all possible regimens (47,520 options tested) yields an grade 3/4 fraction of 89% (28% vs. 89%; p<3.9E-120). Furthermore, the average duration of grade 3/4 neutropenia, decreased notably from 21% of the treatment period without G-CSF, to 3% when G-CSF was optimally administered. Additionally, it was found that day 7 post-docetaxel is the optimal day for G-CSF application (98% of the optimal cases) and the duration for G-CSF administration at day 7 should be three days (94% of the optimal cases). Furthermore, 72% of the optimal schedules included only G-CSF doses of 60-150 μg/day, which are relatively low with regards to the standard dose which is about 300 μg/day (or 5 μg//kg/day).

The standard G-CSF support therapy to Docetaxel Q21D 100 mg/m² is significantly worse than the optimal one and only slightly better than schedules without G-CSF. The standard G-CSF support therapy starts usually one day following the administration of the chemotherapy and is comprised of G-CSF 300 μg/day dose for five consecutive days. Under standard G-CSF therapy 100% of the population is expected to have grade 3/4 neutropenia; moreover, the entire population reaches grade 4 as opposed to schedules without G-CSF support, where only 44% reach grade 4. Overall, the standard G-CSF schedule shortens the duration at grade 3/4 from 21% to 17% of the overall treatment duration, in comparison to no G-CSF, but still this expected result is much greater than the 3% under the optimal regimen (Table 3).

Note that PrediTox predicts much higher occurance of grade 3/4 neutropenia than those observed clinically. This difference can be explained by the difference in sampling frequency between the PrediTox simulator and clinical practice. Since sampling is sparse in clicical practice, there may be many toxic episodes which go undetected. In contrast, the PredTox simulator checks once every 6 hours, and considers a continuous 24 hours in grade 3 or 4 as a grade 3 or 4 episode respectively.

TABLE 3
Tri-weekly docetaxel simulation results
with different G-CSF schedules

| Docetaxel | | | | | Grade 3/4 Neutropenia |
|---|---|---|---|---|---|
| Dose | | Neutropenia Grades | | | Duration [Average % |
| [mg/m²] | G-CSF | [% of population]* | | | of the treatment |
| Q21D | Schedule | G0/1/2** | G3 | G4 | period at G3/4] |
| 100 | None | 0 | 56 | 44 | 21 |
| | Standard*** | 0 | 0 | 100 | 17 |
| | Optimal | 72 | 26 | 2 | 3 |
| | 47,520 | 11 | 23 | 66 | 14 |
| | options | | | | |
| 125 | None | 0 | 39 | 61 | 24 |
| | Optimal | 56 | 33 | 11 | 4 |
| | 47,520 | 5 | 21 | 74 | 17 |
| | options | | | | |
| 150 | None | 0 | 18 | 82 | 25 |
| | Optimal | 35 | 45 | 20 | 6 |
| | 47,520 | 3 | 16 | 81 | 17 |
| | options | | | | |

*The grade is determined if the simulation of the patient spent at least 24 hours. The population's baselines are evenly distributed in the range of 2,000-10,000/μl (in increments of 150).
**G0/1/2: Grade 0 or garde1 or grade 2.
***Standard G-CSF support therapy to Docetaxel Q21D 100 mg/m² starts usually one day following the administration of Docetaxel and comprises with 300 μg/day G-CSF for five days.

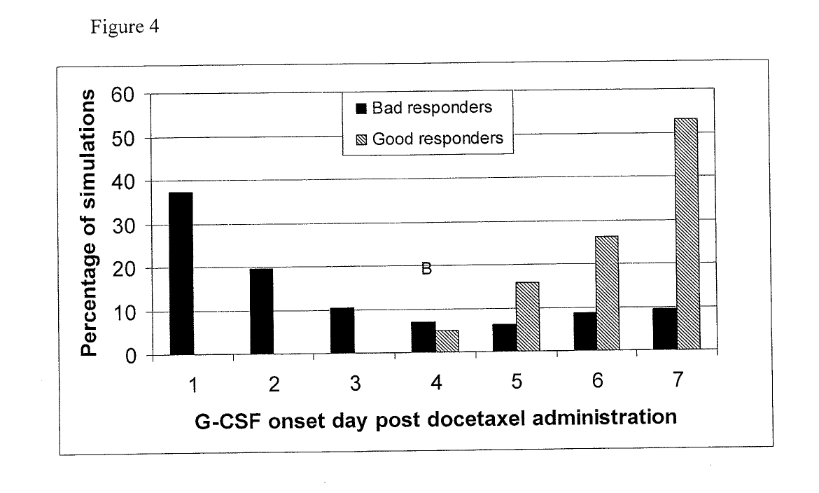

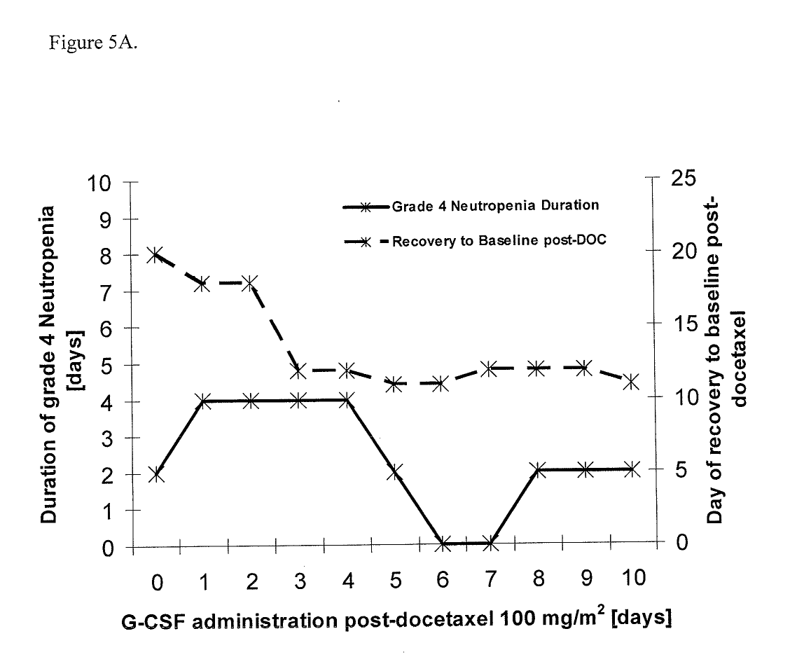

To emphasis the importance of G-CSF timing, we compared the distribution of G-CSF administration day between two simulation subsets from all the possible G-CSF combinations (all with docetaxel 100 mg/m² tri-weekly)—the first included simulations with a maximal grade 4 neutropenia and the grade 3/4 neutropenia duration is more than 4 days (N=1,460; noted as “bad responders”). The second, included simulations that resulted with a maximal grade 2 neutropenia (N=1,074; noted as “good responders”). FIG. 4 shows that the good responders were those with G-CSF administration mainly on days 6-7 post-docetaxel. In contrast, the bad responders were administered with G-CSF mainly on days 1-2 post-docetaxel.

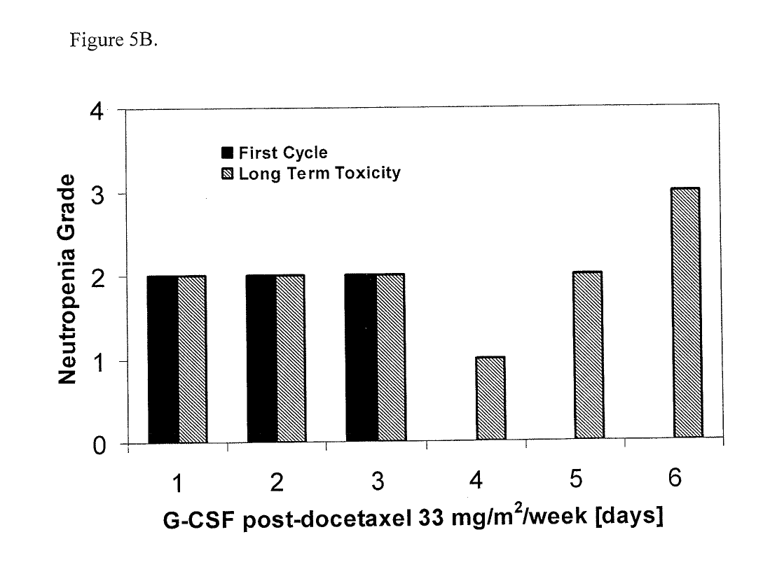

The effect of G-CSF support on docetaxel-induced neutropenia was simulated using the population model, G-CSF dose ranging from 30-480 μg/day, and application day varying from day 1-8 post-docetaxel. Results show that, if optimally timed, 6-7 days post-docetaxel, a dose of 60 μg/day suffices for improving grade 4 neutropenia, which was caused by 75mg/m² tri-weekly docetaxel (FIG. 5A). Higher G-CSF doses result in undesirable leukocytosis (>30,000 neutrophils/μl).

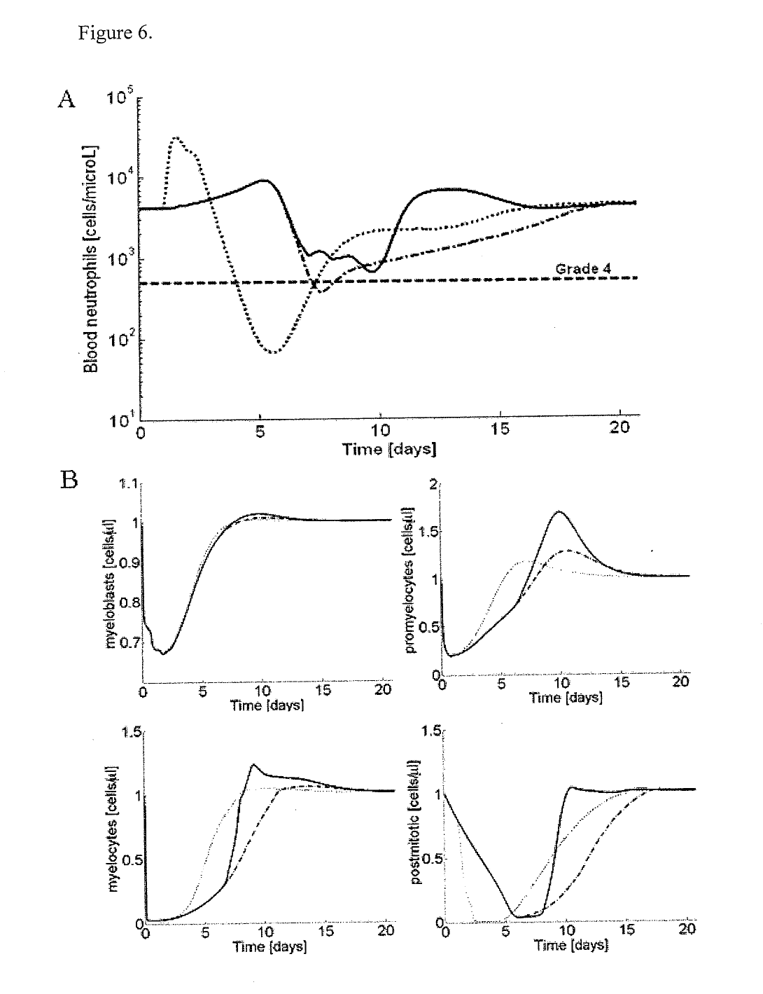

These simulation results suggest that the timing of G-CSF application is crucial for its efficacy, and that wrong timing may increase docetaxel-caused neutropenia, rather than alleviating it. For example, a regimen of 60 μg/day G-CSF, administered QD×3, one day post-docetaxel, 75 mg/m² causes the BM post-mitotic neutrophil reservoir to be rapidly mobilized into blood, followed, ca. 4 days later, by a radical blood neutrophil depletion (grade 4 neutropenia), recovery to baseline occurring at day 18. In contrast, in our simulations, when G-CSF was added 6 days post-docetaxel, neutrophil counts decreased gradually and moderately, reaching only grade 3 neutropenia with complete recovery to baseline at day 11 (FIG. 6). Similar results are expected when G-CSF is applied on the seventh day post-docetaxel.

### Optimal G-CSF Administration Allows to Increase Docetaxel Dose Intensity

Increasing docetaxel's dose intensity may result in a better efficacy but compromises the drug's toxicity (10). To assess toxicity of higher docetaxel doses than the approved 33mg/m²/week, we simulated various G-CSF schedules (30-480 μg/day) with weekly, bi-weekly and tri-weekly docetaxel, 25-125 mg/m²/week. Patients' baseline neutrophil counts varied from 2,000-10,000 neutrophils/μl. For each simulated combination schedule, we evaluated the nadir level and its timing, and recovery time to baseline. The results below are independent of the patient's neutrophil baseline.

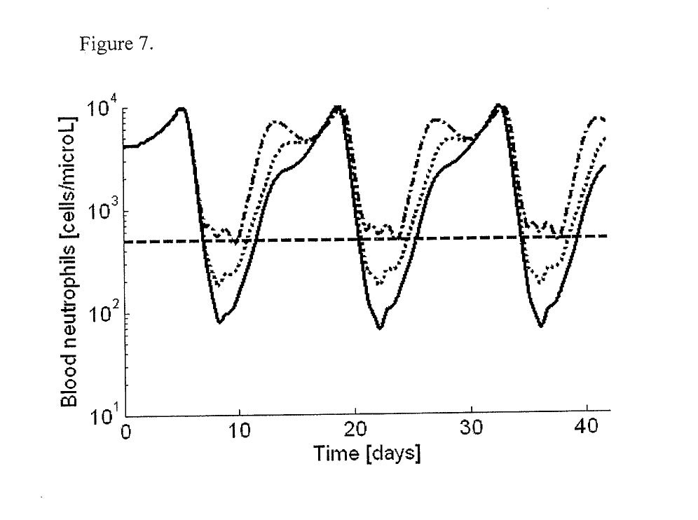

Our results show that when docetaxel is applied alone, intensity of 50 mg/m²/week or higher causes grade 4 neutropenia in the weekly, bi- and tri-weekly regimens, and an incomplete recovery to baseline in the bi- and tri-weekly regimens. We examined the effect of combining high intensity docetaxel with the optimal G-CSF schedule (see above), namely 60 μg/day, QD×3, four days post-weekly docetaxel, 50 mg/m², or six days post-docetaxel, in the bi- and tri-weekly regimens. Results suggest safety improvement to grade 3 neutropenia in the weekly regimen, and grade 4 neutropenia with sufficient recovery to baseline for the bi-weekly 100mg/m² (FIG. 7) and tri-weekly 150 mg/m².

Weekly docetaxel doses, 67 mg/m² or higher, are shown in our simulations to be too toxic even with G-CSF support, causing grade 4 neutropenia and an incomplete recovery to baseline. Acceptable recovery is expected in the equivalent dose intensity of 150 mg/m² bi-weekly and 225 mg/m² tri-weekly, but not in higher dose intensities (bi-weekly FIG. 7 and also tri-weekly).

These results indicate that docetaxel dose intensity can be increased by 50%, if supported by G-CSF, applied QD×3, on days four in the weekly regimen, or on days six-seven in the bi- and tri-weekly regimens. Although grade 4 neutropenia may still occur, an adequate recovery to baseline is predicted.

Increasing docetaxel's dose intensity results in a better efficacy but compromises drug's toxicity (9). To assess dose intensities higher than the approved 33 mg/m²/week with manageable neutropenia, we simulated higher docetaxel intensities, in weekly, bi- and tri-weekly regimens electively supported by G-CSF. PrediTox covers over 650,000 different initial conditions, and the results of the docetaxel/granulopoiesis model predictions under 0, 60, 150, 240, 300, 480 μg/day G-CSF at different onset post-60, 75, 100, 152 and 150 mg/m² tri-weekly docetaxel, or at different onset post-40, 50, 67, 83 and 100 mg/m² bi-weekly docetaxel, ranging from day 1 to 7 post-docetaxel, for 1 to 5 days, or at different onset post-20, 25, 33, 42 and 50 100 mg/m² weekly docetaxel, ranging from day 1 to 4 post-docetaxel, for 1-3 day, assuming a uniform neutrophil baseline distribution in the range of 2,000-10,000 neutrophils/μl (in increments of 150) as an input, over treatment periods of 3, 6, 12, 18 and 36 weeks.

Weekly administration of 33 mg/m² docetaxel alone yields low number of cases with severe neutropenia (Table 4). Moreover, neither all (16,470) possible G-CSF combinations nor the optimal schedules differed significantly in the grade 3/4 neutropenia cases from the simulations without G-CSF. Importantly, almost all of the optimal schedules did not involve G-CSF application (78% of the simulations). These results strengthen the unnecessary administration of G-CSF in the weekly docetaxel regimen of doses up to 33 mg/m²/week.

TABLE 4
Weekly docetaxel simulation results with G-CSF schedules

| Docetaxel | | | | | Grade 3/4 Neutropenia |
|---|---|---|---|---|---|
| Dose | | Neutropenia Grades | | | Duration [Average % |
| [mg/m²] | G-CSF | [% of population]* | | | of the treatment |
| Q7D | Schedule | G0/1/2** | G3 | G4 | period at G3/4] |
| 33 | None | 98 | 11 | 0 | 2 |
| | Optimal | 97 | 3 | 0 | 0 |
| | 16,470 | 70 | 24 | 6 | 6 |
| | options | | | | |
| 42 | None | 54 | 39 | 16 | 13 |
| | Optimal | 75 | 23 | 2 | 3 |
| | 16,470 | 32 | 44 | 24 | 16 |
| | options | | | | |
| 50 | None | 13 | 27 | 60 | 30 |
| | Optimal | 48 | 40 | 12 | 11 |
| | 16,470 | 7 | 37 | 56 | 29 |
| | options | | | | |

*The grade is determined if the simulation of the patient spent at least 24 hours. The population's baselines are evenly distributed in the range of 2,000-10,000/μl (in increments of 150).
**G0/1/2: Grade 0 or garde1 or grade 2.

When increasing docetaxel dose to 42 mg/m²/week without G-CSF, 46% of the simulations resulted in grade 3/4 neutropenia vs. 25% of those with optimal G-CSF schedules (P<3.9E-6). The optimal G-CSF onset was on days 3-4 post-docetaxel for 1-3 days, 60 - 480 μg/day.

Further increase of the weekly docetaxel dose to 50 mg/m²/week resulted with 87% grade 3/4 neutropenia without G-CSF vs. 52% (P<6.6E-17) with the optimal G-CSF schedules, administered on days 3-4 post-docetaxel for 2-3 days. The grade 3/4 neutropenia duration with the optimal schedules was on average only 11% of the treatment period in comparison to 30% of the time without G-CSF.

Increasing the tri-weekly docetaxel dose from 100 to 125 and 150 mg/m², without G-CSF application, resulted in increase of grade 4 from 44% to 61% and 82%, respectively (all those regimens predict that 100% of the patients reach grade 3/4) (Table 3). The optimal G-CSF schedules, decreased grade 3/4 neutropenia cases to 44% and 65% for the 125 and 150 mg/m² doses, respectively (P<2.1E-33). The average duration at grade 3/4 of these regimens is 24% of the treatment periods for 125 mg/m² and 25% for the150 mg/m² doses. Under optimal regimens, the average grade 3/4 neutropenia duration was only 4% and 6%, respectively (Table 3). This means, for example, that over a treatment of 2 cycles, grade 3/4 neutropenia occurs only for ˜2 days.

Note that for both 125 and 150 mg/m² doses docetaxel, all (47,520) G-CSF combination schedules resulted with a similar number of grade 3/4 neutropenia cases (95 and 97%) just a little higher than with no G-CSF administration (89%). However, when the percentage if the population that reach grade 4 are examined an increase is seen from 66% to 74% and 81 for 100, 125 and 150 mg/m².

The optimal G-CSF onset with docetaxel 125 mg/m² ranged from days 4-7 post-docetaxel, mainly on days 6-7, for 3-4 consecutive days (84% of the cases). Specifically, G-CSF doses of 60-240 μg/day at those schedules were 61% of the cases. The optimal G-CSF onset with docetaxel dose of 150mg/m², ranged from days 4-7, where 67% of the cases on days 6-7 for 3-4 days and 60-480 μg/day of G-CSF. These observations consistently support applying G-CSF on days 6-7 in the tri-weekly docetaxel schedule, and that G-CSF dose can be increased as docetaxel dose is intensified above 100 mg/m² to 125 and 150 mg/m², with expected neutropenia that is less sever than the treatments in clinical practice either without G-CSF or with the standard G-CSF protocol.

One promising regimen is the bi-weekly docetaxel administration with an optimal G-CSF timing (Table 5). Simulated bi-weekly docetaxel doses of 67, 83 and 100 mg/m² without G-CSF, resulted with grade 3/4 neutropenia cases of 54%, 87% and 100%, with average neutropenia duration of 18%, 25%, 30% of the treatment periods, respectively. The optimal G-CSF schedules, resulted with a significant (P<1.15E-33) decrease in the grade 3/4 neutropenia percentage of the population to 6% 17% and 31%, respectively, with average grade 3/4 neutropenia duration of maximally 5% of the treatment period (Table 5). Analyzing the percentage of the population that reach grade 4, a remarkable effect to of the optimal G-CSF regimen is seen

The main G-CSF onset of the optimal G-CSF schedules was on days 6-7 post-docetaxel, for 3-4 days (89%-100% of the cases in the three docetaxel doses), and with this timing and low G-CSF dose of 60-150 μg/day being 50%-74% of the optimal cases.

TABLE 5
Bi-weekly docetaxel simulation results with G-CSF schedules

| Docetaxel | | | | | Grade 3/4 Neutropenia |
|---|---|---|---|---|---|
| Dose | | Neutropenia Grades | | | Duration [Average % |
| [mg/m²] | G-CSF | [% of population]* | | | of the treatment |
| Q14D | Schedule | G0/1/2** | G3 | G4 | period at G3/4] |
| 67 | None | 46 | 39 | 15 | 18 |
| | Optimal | 94 | 6 | 0 | 0 |
| | 47,520 | 26 | 30 | 44 | 13 |
| | options | | | | |
| 83 | None | 13 | 56 | 31 | 25 |
| | Optimal | 83 | 17 | 0 | 2 |
| | 47,520 | 16 | 26 | 59 | 17 |
| | options | | | | |
| 100 | None | 0 | 56 | 44 | 30 |
| | Optimal | 69 | 28 | 3 | 5 |
| | 47,520 | 10 | 23 | 67 | 20 |
| | options | | | | |

*The grade is determined if the simulation of the patient spent at least 24 hours. The population's baselines are evenly distributed in the range of 2,000-10,000/μl (in increments of 150).
**G0/1/2: Grade 0 or garde1 or grade 2.

A further inspection of the bi-weekly docetaxel regimen showed that when applying the optimal G-CSF schedules, the recovery of neutrophils to baseline level occurs at day 11 (FIG. 7). This fast recovery allows the subsequent docetaxel dosing at day 14, thus increasing the total docetaxel dose intensity.

In summary, using the combined docetaxel/granulopoiesis model, we are able to predict the maximum tolerable intensified dose for docetaxel/G-CSF treatment for the individual MBC patients with three docetaxel accepted regimens.

### Maximizing Individual Safety of Intensified Docetaxel Regimens

To show that different individual biological make-ups dictate different safety constraints and, hence, different personalized treatments, we adapted our model to individually describe each of two patients. These were taken from the study population, and differed in the response to docetaxel. For each patient, our simulations identified a personalized treatment schedule of maximum docetaxel dose intensity, yielding no more than grade 3 neutropenia.

First, we simulated the less susceptible, Patient1, under various docetaxel intensities supported by G-CSF, 60 μg/day, 6 days post-docetaxel, for three days. The model predicts that under a dose intensity of 50 mg/m²/week this patient is expected to suffer no neutropenia by the weekly and bi-weekly administration and a grade 1 neutropenia by the tri-weekly schedule. Increasing docetaxel intensity to 125 mg/m²/week, may result in grade 3 neutropenia after each cycle of the bi-weekly and the equivalent tri-weekly regimens, while weekly administration is expected to result in grade 3 and 4 neutropenia only after the first and second cycles, respectively. A further increase in docetaxel intensity will result in grade 4 neutropenia for all regimens.

Simulating the same treatment in the model adjusted to mimic the more susceptible, Patient2, predicts that all schedules of intensity, higher than 50 mg/m²/week, may result in grade 4 neutropenia, in contrast to maximum grade 3 neutropenia predicted for lower dose intensities.

Thus, using the combined docetaxel/granulopoiesis model, we are able to pinpoint personalized docetaxel/G-CSF regimens for the individual MBC patient.

### Implications of the Invention

In this work we clinically validated a computational methodology for predicting chemotherapy-induced neutropenia in MBC patients, based on a mathematical granulopoiesis population model (24). The ability to predict nadir's timing and neutropenia grade prior to treatment is of high clinical importance. Therefore, it is encouraging that the model proves accurate in predicting grade 3/4 neutropenia in most patients that experienced it and in no other patients (positive predictive value of 86%; kappa=0.69), and is highly precise in predicting the individual patient's nadir (r=0.99). Indeed, due to the relatively infrequent measurements of blood counts in the clinic, the lowest observable neutrophil count is not necessarily the true nadir. However, our model was validated for its high precision in predicting all the recorded counts around nadir, including the true nadir when this was recorded (FIG. 2A-D).

An important advantage of the model lies in its ability to use clinical data for evaluating characteristic population parameters, which cannot be retrieved from literature. After estimating these parameters using the training set, and confirming model prediction accuracy by the validation set, the generalization of the model to the entire population is still to be confirmed. Mixing patient populations from different origins in the training set, as we did here, or using large data sets are methods used for sustaining model generality. In our case, the model was validated by independent data, including docetaxel plasma measurements (30) and of a phase I clinical trial results (31). This validation suggests that little adjustment is necessary for adapting the model to different cancer patient populations (FIG. 8).

### Optimal Schedule of G-CSF Administration as Support Therapy to Chemotherapy

Simulation results clearly showed that the success of G-CSF crucially depends on the time of its administration. G-CSF 6-7 days post tri-weekly docetaxel improves docetaxel-afflicted neutropenia, whereas its administration immediately after chemotherapy will yield worse results than with docetaxel alone (FIG. 6). Meisenberg et al., studied this problem in non-human primates and showed that G-CSF treatment one day post-chemotherapy speeds up and aggravates neutropenia, as predicted by our model (FIG. 6A). Applying a G-CSF continuously , these authors observe a relatively fast recovery to baseline (33). Indeed, simulating Meisenberg et al.'s treatment schedule showed grade 4 neutropenia with a fast recovery to baseline at day 10.

These results are explained, as follows. It is known that G-CSF has two major effects on granulopoiesis: (i) acceleration of neutrophil production, and (ii) rapid release of neutrophils from BM reservoirs to blood (24). Being a cell-cycle specific drug, docetaxel damages the early stages in the neutrophil development pipeline (34, 35). Our model simulations show that cells from the undamaged post-mitotic compartment and BM neutrophil reservoirs are gradually mobilized into blood to compensate for the short-lived circulating neutrophils. However, administration of G-CSF immediately following docetaxel, mobilizes the neutrophil reservoirs into blood, prior to the docetaxel-induced nadir. Now the depleted BM reservoirs can no longer compensate for blood neutrophil shortage, and as a consequence, the nadir is more profound than that without G-CSF (FIG. 6A). In contrast, when G-CSF is applied 6-7 days post-docetaxel, the release of neutrophils from the post-mitotic BM reservoir overlaps, and hence, moderates the effect of docetaxel's damage to BM progenitors. Moreover, the recovery to baseline is more rapid, due to a more efficient stimulatory effect of G-CSF on neutrophil production once BM cell production is partly recovered (FIG. 6B). This analysis of the model reveals a mechanism that may be relevant to other chemotherapy induced neutropenia: the timing in which the administration of the supportive therapy (e.g., G-CSF, pegylated G-CSF) is most effective is at the nadir, or in a range of a day or two around the nadir. Therefore, for a chemotherapy agent, once the nadir of neutrophil level in the plasma is determined, the timing of the supportive therapy can be set to this day or around this day. For example, for the chemotherapy vinflunine the nadir of neutrophil level in the plasma is usually at day 20, so this could serve as preferred day for administration of G-CSG or pegylated G-CSF. For the chemotherapy Irinotecan for instance, the neutropenia nadir is on day 9, which may serve at the day for the administration of the supportive therapy.

Irrespective of the patient's neutrophils baseline, maximum improvement of neutrophil counts and their fastest recovery to baseline is predicted for a G-CSF regimen, 60 μg/day, administered 6-7 days post-docetaxel, QD×3, in the bi- and tri-weekly docetaxel dosings. This allows doubling the approved docetaxel dose intensity, 33mg/m²/week. Indeed, increasing docetaxel dose up to 145 mg/m², resulted in acceptable neutropenia, as reported in (36).

It was shown previously and supported by our simulations (FIG. 3) that a weekly docetaxel regimen, 33 mg/m², is less myelotoxic than the comparable bi- and tri-weekly schedules, and progression-free and overall survival were reported similar for the weekly and tri-weekly regimens (5-9). Simulations of a vascular tumor growth model, prospectively validated in docetaxel treated, xenografts of mesenchymal chondrosarcoma patient's biopsies, indicate superiority for the weekly regimen for patients with intensive tumor angiogenesis (37). Note, though, that the bi-weekly regimen might be finally elected since the once-weekly regimen may prove less convenient. However, in order to systematically reduce nuetropenia or alternatively use dose intense regimens of chemotherapy, which nuetropenia is the dose limiting toxicity (DLT), the use of our model and its predictions is essential.

Our results suggest that increasing the weekly docetaxel dose to 50 mg/m² may lead to grade 4 neutropenia, as supported by data from a dose escalation study, where 14 patients received weekly docetaxel, 40-45 mg/m², for three consecutive weeks in cycles of four weeks. Half of these patients suffered grade 3/4 neutropenia, but none of the patients receiving 30-35 mg/m2/week (38). We suggest, then, that although G-CSF is not mandatory in the approved weekly docetaxel regimen, it should be considered when higher weekly doses are applied, preferably, for 2-3 consecutive days, timed 4 days post-docetaxel (FIG. 5B).

### Personalized PD Model.

Personalized PD parameters were estimated for two patients who differed in the effect of 100 mg/m² tri-weekly docetaxel on neutrophil counts and their baseline characteristics (respectively for patient 1 and 2—ages: 53, 41; body mass index: 21.5, 24.1; body surface area: 1.67, 1.59. Time from prior chemotherapy: 1 week, 25 months; Metastases number: 4, 3; Metastases location: lymph nodes and liver, liver; baseline neutrophil count: 5,400, 6,200; neutrophils at nadir: 400, 200; nadir day: 7, 7). The personalized models were simulated under different schedules of docetaxel and G-CSF.

### PrediTox a Tool for the Individual and General Prediction of Neutropenia

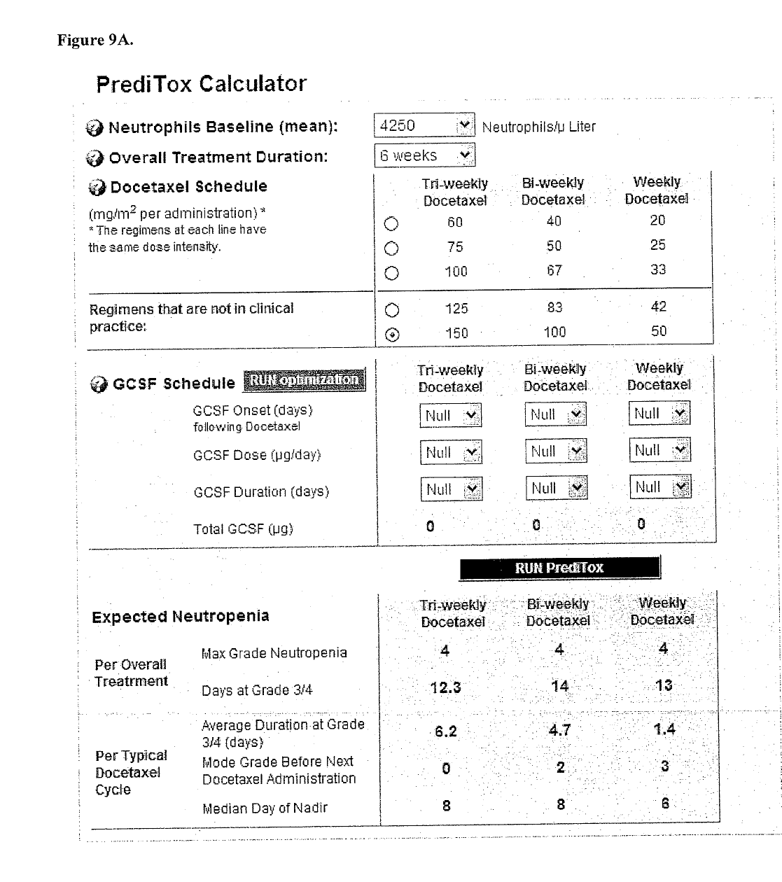

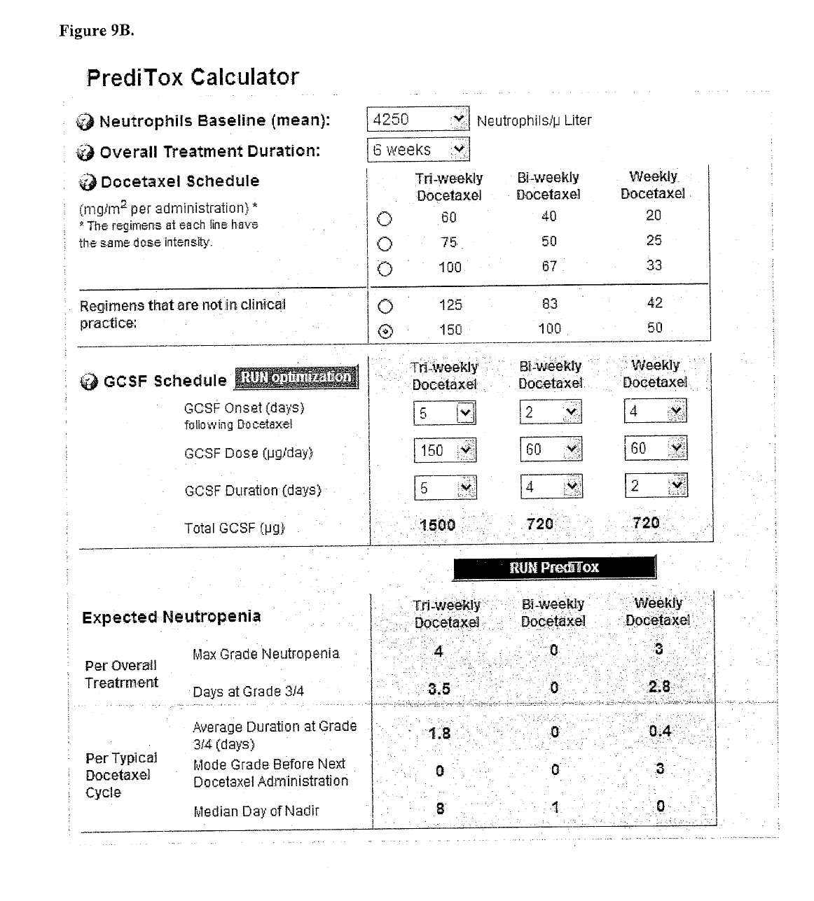

Despite the vast experience with docetaxel (3, 4, 7-9, 38, 39) and G-CSF (10, 40), there is still no agreement on the desired G-CSF schedules, as such agreement may only be reached by many laborious and expensive clinical trials. Moreover, individual patients' nadir time cannot be predicted. These clinical problems can be addressed by our model, which differs from simple PK/PD models in accounting for the biological system's dynamics—a prerequisite for predicting long-term patient response, such as the nadir timing. As it allows for an elaborate BM dynamics, our model enables to predict response over continuous treatment periods, in contrast to other models, predicting response over a single treatment cycle (17, 20, 28). Moreover, our model is unique in being able to determine the optimal timing of G-CSF application, since it embeds G-CSF long-term effects on the proliferating and maturing granulocyte BM compartments (21, 24). Importantly, our model differs from previously published models in its ability to tailor individual chemotherapy/G-CSF combination schedule prior to treatment. Therefore, we have developed PrediTox, a tool that can be implemented in internet web site or handheld machine/calculator, towards routine implementation of mathematical models in oncology schedule optimization (FIG. 9). An interactive version of the model can found in http://www.preditox.com (or temporary in http://www.preditox.com/Default.asp).

### Influence of Inter-Patient PK Variability on Prediction Accuracy.

We examined the influence of CYP3A inter-individual variability (27) on the model's prediction accuracy by varying the PK parameter mostly affected by this variability, namely docetaxel clearance. Accordingly, we created 100 patient models, representing each patient in our validation population, except for the docetaxel clearance parameter, which was randomly taken from the observed range of 5.4-29.1 L/hr/m² (27). We simulated each “new” patient with the combined docetaxel/granulopoiesis model and calculated the difference in the prediction accuracy of nadir timing and grade 3/4 neutropenia, between the general population model, assuming average PK parameters, and the model assuming variable PK. Prior chemotherapy, performance status, ethnic origin are also factors that are known to affect PK parameters.

### The Effect of CYP3A Genetic Variations

To evaluate the robustness of the population model, we checked how variability in the enzyme CYP3A would influence the accuracy of model predictions. As variability in CYP3A activity directly affects docetaxel clearance (27), we simulated our model, replacing the population average docetaxel clearance parameter by randomly assigned clearance values, normally distributed within the CYP3A-affected clinically observed range (5.4-29.1 Uhr/m²; ref. 27). The new model predictions remain largely unchanged when CYP3A-induced PK variability is incorporated. Predicted nadir on day 7.86±0.27 under population average clearance, becomes day 7.65±1.8E-15 post-docetaxel, under CYP3A variability, and grade 3/4 neutropenia duration, being 4.1±2.3 days under population average clearance, becomes 5.19±2.16 days under CYP3A variability. The positive predictive value is slightly reduced, to 70%±5.3% and the negative predictive value was hardly changed (87.5%±10.9%).

Taken together, the above results demonstrate high accuracy of the population docetaxel/granulopoiesis model in predicting neutrophil counts following docetaxel treatment. The generalizability of our model is reinforced by its demonstrated robustness to the introduction of PK variability.

Model predictions are robust to variability in docetaxel clearance due to variable CYP3A activity (27). Our simulation results suggest that the introduction of CYP3A-induced PK variability hardly affects the predicted nadir timing and grade 3/4 neutropenia duration, but slightly reduces the positive predictive value. Based on these results one may conclude that model predictions under the assumption of population average drug clearance are robust to PK variability due to CY3A variability. Other parameters, possibly inducing variable myelotoxicity in cancer patients, include alpha-1 acid glycoprotein (AAG), to which docetaxel binds (28,32), BRCA1/2 (49), etc. When individual measurements of such proteins are available, they can be easily integrated to the model, to further adjust the individual predictions. Information on population distribution of different parameters can also be implemented in the model.

### Predicted Neutropenia Due to Other Chemotherapy and Other Chemo-Supportive Agents

The generalization of the model enables its application to other chemotherapeutic and chemo-supportive agents, including pegylated G-CSF. We have modeled the PK of pegylated G-CSF with only a change of one parameter of the model (its clearance rate) in comparison to not pegylated G-CSF. We have managed to identify conditions where PrediTox PK model the model fits well with experimental pegylated G-CSF PK results. This new configuration of the model predicts the neutropenia of docetaxel when the supportive agent is pegylated G-CSF.

In similar way we may use other colony stimulating factors (CSFs) as Macrophage Colony-Stimulating Factor and Granulocyte Macrophage Colony-Stimulating Factor. With regards to the various chemotherapies, in fact, the model can be applied to each neutropenia causing agent, especially when neutropenia is its dose limiting toxicity (DLT). A few examples for such agents are: Doxorubicin, Temozolomide, Taxol/ Paclitaxel, Irinotecan and Carboplatin.

### REFERENCES

(1) Cortes, J.E. & Pazdur, R. Docetaxel. J Clin Oncol 13, 2643-55 (1995).

(2) Montero, A., Fossella, F., Hortobagyi, G. & Valero, V. Docetaxel for treatment of solid tumours: a systematic review of clinical data. Lancet Oncol 6, 229-39 (2005).

(3) Oishi, T. et al. A feasibility study on biweekly administration of docetaxel for patients with recurrent ovarian cancer. Gynecol Oncol 90, 421-4 (2003).

(4) Vazquez, S. et al. Biweekly docetaxel as second-line chemotherapy of patients with advanced non-small cell lung cancer: a phase II study of the Galician Lung Cancer Group (GGCP 006-00). Anticancer Drugs 15, 489-94 (2004).

(5) Mey, U., Gorschluter, M., Ziske, C., Kleinschmidt, R., Glasmacher, A. & Schmidt-Wolf, I. G. Weekly docetaxel in patients with pretreated metastatic breast cancer: a phase II trial. Anticancer Drugs 14, 233-8 (2003).

(6) Rivera, E. et al. Phase 3 study comparing the use of docetaxel on an every-3-week versus weekly schedule in the treatment of metastatic breast cancer. Cancer 112, 1455-61 (2008).

(7) Schuette W, Nagel S, Blankenburg T, Lautenschlaeger C, Hans K, Schmidt E W, Dittrich I, Schweisfurth H, von Weikersthal L F, Raghavachar A, Reissig A, Serke M. Phase III study of second-line chemotherapy for advanced non-small-cell lung cancer with weekly compared with 3-weekly docetaxel. J Clin Oncol. 2005 Nov. 20; 23(33):8389-95.

(8) Gervais R, Ducolone A, Breton J L, Braun D, Lebeau B, Vaylet F, Debieuvre D, Pujol J L, Tredaniel J, Clouet P, Quoix E. Phase II randomised trial comparing docetaxel given every 3 weeks with weekly schedule as second-line therapy in patients with advanced non-small-cell lung cancer (NSCLC). Ann Oncol. 2005 January; 16(1):90-6.

(9) Gridelli C, Gallo C, Di Maio M, Barletta E, Illiano A, Maione P, Salvagni S, Piantedosi F V, Palazzolo G, Caffo O, Ceribelli A, Falcone A, Mazzanti P, Brancaccio L, Capuano M A, Isa L, Barbera S, Perrone F. A randomised clinical trial of two docetaxel regimens (weekly vs 3 week) in the second-line treatment of non-small-cell lung cancer. The DISTAL 01 study. Br J Cancer. 2004 Dec. 13; 91(12):1996-2004.

(10) Paciucci, P. A., Raptis, G., Bleiweiss, I., Weltz, C., Lehrer, D. & Gurry, R. Neo-adjuvant therapy with dose-dense docetaxel plus short-term filgrastim rescue for locally advanced breast cancer. Anticancer Drugs 13, 791-5 (2002).

(11) Gridelli, C. et al. Single agent docetaxel plus granulocyte-colony stimulating factor (G-CSF) in previously treated patients with advanced non small cell lung cancer. A phase II study and review of the literature. Anticancer Res 20, 1077-84 (2000).

(12) Smith T J, Khatcheressian J, Lyman G H, Ozer H, Armitage J O, Balducci L, Bennett C L, Cantor S B, Crawford J, Cross S J, Demetri G, Desch C E, Pizzo P A, Schiffer C A, Schwartzberg L, Somerfield M R, Somlo G, Wade J C, Wade J L, Winn R J, Wozniak A J, Wolff A C. 2006 update of recommendations for the use of white blood cell growth factors: an evidence-based clinical practice guideline. J Clin Oncol. 2006 Jul. 1; 24(19):3187-205. Epub 2006 May 8.

(13) Schmitz, S., Franke, H., Brusis, J. & Wichmann, H. E. Quantification of the cell kinetic effects of G-CSF using a model of human granulopoiesis. Exp Hematol 21, 755-60 (1993).

(14) Schmitz, S., Loeffler, M., Jones, J. B., Lange, R. D. & Wichmann, H. E. Synchrony of bone marrow proliferation and maturation as the origin of cyclic haemopoiesis. Cell Tissue Kinet 23, 425-42 (1990).

(15) Mackey, M. C., Aprikyan, A. A. & Dale, D. C. The rate of apoptosis in post mitotic neutrophil precursors of normal and neutropenic humans. Cell Prolif 36, 27-34 (2003).

(16) Ostby, I., Rusten, L. S., Kvalheim, G. & Grottum, P. A mathematical model for reconstitution of granulopoiesis after high dose chemotherapy with autologous stem cell transplantation. J Math Biol 47, 101-36 (2003).

(17) Friberg, L. E., Henningsson, A., Maas, H., Nguyen, L. & Karlsson, M. O. Model of chemotherapy-induced myelosuppression with parameter consistency across drugs. J Clin Oncol 20, 4713-21 (2002).

(18) Engel C, Scholz M, Loeffler M. A computational model of human granulopoiesis to simulate the hematotoxic effects of multicycle polychemotherapy. Blood. 2004 Oct. 15; 104(8):2323-31. Epub 2004 Jun. 29.

(19) Shochat, E., Stemmer, S. M. & Segel, L. Human haematopoiesis in steady state and following intense perturbations. Bull Math Biol 64, 861-86 (2002).

(20) Shochat E, Rom-Kedar V. Novel strategies for granulocyte colony-stimulating factor treatment of severe prolonged neutropenia suggested by mathematical modeling. Clin Cancer Res. 2008 Oct. 15; 14(20):6354-63.

(21) de Haas, M., Kerst, J. M., van der Schoot, C. E., Calafat, J., Hack, C. E., Nuijens, J. H., Roos, D., van Oers, R. H., von dem Borne, A. E., 1994. Granulocyte colony-stimulating factor administration to healthy volunteers: analysis of the immediate activating effects on circulating neutrophils. Blood 84, 3885-3894.

(22) Shimazaki, C., Uchiyama, H., Fujita, N., Araki, S., Sudo, Y., Yamagata, N., Ashihara, E., Goto, H., Inaba, T., Haruyama, H., 1995. Serum levelsof endogenousand exogenous granulocyte colony-stimulating factor afterautologous blood stem cell transplantation. Exp. Hematol. 23, 1497-1502.

(23) Price, T. H., Chatta, G. S. & Dale, D. C. Effect of recombinant granulocyte colony-stimulating factor on neutrophil kinetics in normal young and elderly humans. Blood 88, 335-40 (1996).

(24) Vainstein, V., Ginosar, Y., Shoham, M., Ranmar, D. O., Ianovski, A. & Agur, Z. The complex effect of granulocyte colony-stimulating factor on human granulopoiesis analyzed by a new physiologically-based mathematical model. J Theor Biol 234, 311-27 (2005).

(25) Peter L. Bonate “Pharmacokinetic-Pharmacodynamic Modeling and Simulation”.

(26) Kim H. Esbensen, Dominique Guyot, Frank Westad, Lars P. Houmøller “Multivariate data analysis”.

(27) Hirth J, Watkins P B, Strawderman M, Schott A, Bruno R, Baker L H. The effect of an individual's cytochrome CYP3A4 activity on docetaxel clearance. Clin Cancer Res. 2000 April; 6(4):1255-8.

(28) Ozawa, K., Minami, H. & Sato, H. Population pharmacokinetic and pharmacodynamic analysis for time courses of docetaxel-induced neutropenia in Japanese cancer patients. Cancer Sci 98, 1985-92 (2007).

(29) Skomorovski, K. et al. New TPO treatment schedules of increased safety and efficacy: pre-clinical validation of a thrombopoiesis simulation model. Br J Haematol 123, 683-91 (2003).

(30) van Zuylen L, Sparreboom A, van der Gaast A, van der Burg M E, van Beurden V, Bol C J, Woestenborghs R, Palmer P A, Verweij J. The orally administered P-glycoprotein inhibitor R101933 does not alter the plasma pharmacokinetics of docetaxel. Clin Cancer Res. 2000 April; 6(4):1365-71.

(31) Extra, J. M., Rousseau, F., Bruno, R., Clavel, M., Le Bail, N. & Marty, M. Phase I and pharmacokinetic study of Taxotere (RP 56976; NSC 628503) given as a short intravenous infusion. Cancer Res 53, 1037-42 (1993).

(32) Urien, S., Barre, J., Morin, C., Paccaly, A., Montay, G. & Tillement, J. P. Docetaxel serum protein binding with high affinity to alpha 1-acid glycoprotein. Invest New Drugs 14, 147-51 (1996).

(33) Meisenberg, B. R., Davis, T. A., Melaragno, A. J., Stead, R. & Monroy, R. L. A comparison of therapeutic schedules for administering granulocyte colony-stimulating factor to nonhuman primates after high-dose chemotherapy. Blood 79, 2267-72 (1992).

(34) Gueritte-Voegelein, F., Guenard, D., Lavelle, F., Le Goff, M. T., Mangatal, L. & Potier, P. Relationships between the structure of taxol analogues and their antimitotic activity. J Med Chem 34, 992-8 (1991).

(35) Jordan, M. A. & Wilson, L. Microtubules as a target for anticancer drugs. Nat Rev Cancer 4, 253-65 (2004).

(36) Masters, G. A., Brockstein, B. E., Mani, S. & Ratain, M. J. Phase 1 dose escalation study of docetaxel with filgrastim support in patients with advanced solid tumors. Med Oncol 20, 7-12 (2003).

(37) Gorelik B, Ziv I, Shohat R, Wick M, Hankins W D, Sidransky D, Agur Z. Efficacy of weekly docetaxel and bevacizumab in mesenchymal chondrosarcoma: a new theranostic method combining xenografted biopsies with a mathematical model. Cancer Res. 2008 Nov. 1; 68(21):9033-40. Erratum in: Cancer Res. 2008 Dec 1;68(23):10005.

(38) Kouroussis C, Agelaki S, Mavroudis D, Souglakos J, Kakolyris S, Kalbakis K, Vardakis N, Reppa D, Hatzidaki D, Samonis G, Georgoulias V. A dose escalation study of weekly docetaxel in patients with advanced solid tumors. Cancer Chemother Pharmacol. 2000; 46(6):488-92.

(39) Lai C L, Tsai C M, Chiu C H, Wang G S, Su W J, Chen Y M, Perng R P. Phase II randomized trial of tri-weekly versus days 1 and 8 weekly docetaxel as a second-line treatment of advanced non-small cell lung cancer. Jpn J Clin Oncol. 2005 December; 35(12):700-6. Epub 2005 Nov. 22.

(40) Aapro M S, Cameron D A, Pettengell R, Bohlius J, Crawford J, Ellis M, Kearney N, Lyman G H, Tjan-Heijnen V C, Walewski J, Weber D C, Zielinski C; European Organisation for Research and Treatment of Cancer (EORTC) Granulocyte Colony-Stimulating Factor (G-CSF) Guidelines Working Party. EORTC guidelines for the use of granulocyte-colony stimulating factor to reduce the incidence of chemotherapy-induced febrile neutropenia in adult patients with lymphomas and solid tumours. Eur J Cancer. 2006 October; 42(15):2433-53. Epub 2006 Jun. 5. Review.

(41) Brunsvig, P. F., Andersen, A., Aamdal, S., Kristensen, V. & Olsen, H. Pharmacokinetic analysis of two different docetaxel dose levels in patients with non-small cell lung cancer treated with docetaxel as monotherapy or with concurrent radiotherapy. BMC Cancer 7, 197 (2007).

(42) Baker S D, Zhao M, Lee C K, Verweij J, Zabelina Y, Brahmer J R, Wolff A C, Sparreboom A, Carducci M A. Comparative pharmacokinetics of weekly and every-three-weeks docetaxel. Clin Cancer Res. 2004 Mar. 15; 10(6):1976-83.

(43) Engels F K, de Jong F A, Sparreboom A, Mathot R A, Loos W J, Kitzen J J, de Bruijn P, Verweij J, Mathijssen R H. Medicinal cannabis does not influence the clinical pharmacokinetics of irinotecan and docetaxel. Oncologist. 2007 March; 12(3):291-300.

(44) Hurria A, Fleming M T, Baker S D, Kelly W K, Cutchall K, Panageas K, Caravelli J, Yeung H, Kris M G, Gomez J, Miller V A, D'Andrea G, Scher H I, Norton L, Hudis C. Pharmacokinetics and toxicity of weekly docetaxel in older patients. Clin Cancer Res. 2006 Oct. 15; 12(20 Pt 1):6100-5.

(45) Ramanathan R K, Ramalingam S, Egorin M J, Belani C P, Potter D M, Fakih M, Jung L L, Strychor S, Jacobs S A, Friedland D M, Shin D M, Chatta G S, Tutchko S, Zamboni W C. Phase I study of weekly (day 1 and 8) docetaxel in combination with capecitabine in patients with advanced solid malignancies. Cancer Chemother Pharmacol. 2005 April; 55(4):354-60. Epub 2004 Oct. 27.

(46) Witta S E, Gustafson D L, Pierson A S, Menter A, Holden S N, Basche M, Persky M, O'Bryant C L, Zeng C, Baron A, Long M E, Gibbs A, Kelly K, Bunn P A Jr, Chan D C, Pallansch P, Eckhardt S G. A phase I and pharmacokinetic study of exisulind and docetaxel in patients with advanced solid tumors. Clin Cancer Res. 2004 Nov. 1; 10(21):7229-37.

(47) Pazdur R, Newman R A, Newman B M, Fuentes A, Benvenuto J, Bready B, Moore D Jr, Jaiyesimi I, Vreeland F, Bayssas M M, et al. Phase I trial of Taxotere: five-day schedule. J Natl Cancer Inst. 1992 Dec. 2; 84(23):1781-8.

(48) Kroese, D., Porotsky, S. & Rubinstein, R. The Cross-Entropy Method for Continuous Multi-Extremal Optimization. Methodol Comput Appl Probab 8, 383-407 (2006).

(49) Shanley S, McReynolds K, Ardern-Jones A, Ahern R, Fernando I, Yarnold J, Evans G, Eccles D, Hodgson S, Ashley S, Ashcroft L, Tutt A, Bancroft E, Short S, Smith I, Gui G; Royal Marsden NHS Foundation Trust, Barr L, Baildam A, Howell A, Royle G, Pierce L, Easton D, Eeles R. Acute chemotherapy-related toxicity is not increased in BRCA1 and BRCA2 mutation carriers treated for breast cancer in the United Kingdom. Clin Cancer Res. 2006 Dec. 1; 12(23):7033-8.

1. A method of determining an optimal therapeutic regimen for the treatment of cancer with docetaxel comprising:
obtaining data to determine a docetaxel computer model for pharmacodynamics and pharmacokinetics of docetaxel based upon effects of docetaxel administration in vitro and/or in vivo;
creating a docetaxel/ granulopoiesis computer model to predict an optimum treatment regimen for cancer by combination of the docetaxel computer model and granulopoiesis computer model;
determining an optimal therapeutic regimen with docetaxel from the one or more treatment schedules by comparing results of computer simulations from the docetaxel/granulopoiesis computer model for reduced docetaxel-induced toxicity between the one or more treatment schedules.

2. The method of claim 1, wherein the cancer is selected from the group comprising breast cancer, lung cancer, prostate cancer, gastric cancer, head and neck cancer, melanoma, bladder cancer, neuroendocrine cancer, squamous carcinoma, cervical cancer, vulvar cancer, thyroid cancer, pancreatic cancer, renal cancer, esophageal cancer, rectal cancer, penile cancer, lymphoma, multiple myloma, Merkel cell tumors, ovarian cancer or colorectal cancer.

3. The method of claim 1, wherein the docetaxel-induced toxicity is neutropenia.

4. The method of claim 3, wherein the neutropenia is a grade 0, 1, 2, 3, 4 or 3/4 neutropenia.

5. The method of claim 1, wherein the granulopoeisis model accounts for effects of post-docetaxel administration of Granulocyte-Colony Stimulating Factor (G-CSF) or pegylated Granulocyte-Colony Stimulating Factor (peg G-CSF).

6. The method of claim 5, wherein G-CSF or pegylated G-CSF is administered at least 6-7 days post-docetaxel administration.

7. A method for determining optimal therapeutic regimen for the treatment of cancer with docetaxel in combination with Granulocyte Colony-Stimulating Factor (G-CSF) or pegylated Granulocyte Colony-Stimulating Factor (peg G-CSF) comprising:
obtaining data to determine a docetaxel computer model for pharmacodynamics and pharmacokinetics of docetaxel based upon effects of docetaxel administration in vitro and/or in vivo;
creating a docetaxel/granulopoiesis computer model to predict an optimum treatment regimen for cancer by combination of the docetaxel computer model and granulopoiesis computer model;
determining the optimal therapeutic regimen from the one or more treatment schedules by comparing results of the computer simulations from the docetaxel/granulopoiesis computer model for reduced docetaxel-induced toxicity;
wherein the optimal therapeutic regimen comprises administration of docetaxel in combination with G-CSF or pegylated G-CSF.

8. The method of claim 7, wherein the cancer is selected from the group comprising of breast cancer, lung cancer, prostate cancer, gastric cancer, head & neck cancer, melanoma, bladder cancer, neuroendocrine cancer, squamous carcinoma, cervical cancer, vulvar cancer, thyroid cancer, pancreatic cancer, renal cancer, esophageal cancer, rectal cancer, penile cancer, lymphoma, multiple myloma, Merkel cell tumors, ovarian cancer or colorectal cancer.

9. The method of claim 7, wherein the docetaxel/granulopoiesis model is adjusted by at least one factor that may affect the pharmacokinetics of docetaxel, G-CSF, or pegylated G-CSF, comprising: neutrophils baseline, age, gender, alpha 1-acid glycoprotein, prior chemotherapy, performance status, ethnic origin, genetic variations of Cytochrome P450 genes and CYP3A.

10. The method of claim 7, wherein the docetaxel-induced toxicity is neutropenia.

11. The method of claim 10, wherein the neutropenia is a grade 0, 1, 2, 3, 4 or 3/4 neutropenia.

12. The method of claim 7, wherein G-CSF or pegylated G-CSF is administered at least 6-7 days post-docetaxel administration.

13. A system for optimizing a therapeutic regimen for the treatment of cancer with docetaxel, the system comprising:
a docetaxel computer model for pharmacodynamics and pharmacokinetics of docetaxel based upon effects of docetaxel administration in vitro and or in vivo;
a granulopoiesis computer model that includes a process model for cells involved in neutrophil lineage to predict docetaxel-induced toxicity.

14. The system of claim 13, wherein the cancer is selected from the group comprising of breast cancer, lung cancer, prostate cancer, gastric cancer, head & neck cancer, melanoma, bladder cancer, neuroendocrine cancer, squamous carcinoma, cervical cancer, vulvar cancer, thyroid cancer, pancreatic cancer, renal cancer, esophageal cancer, rectal cancer, penile cancer, lymphoma, multiple myloma, Merkel cell tumors, ovarian cancer or colorectal cancer.

15. The system of claim 13, wherein the system is stored in a computer readable medium.

16. The system of claim 13, wherein the system operates over the internet.

17. The system of claim 13, wherein the system is stored in a hand-held calculator.

18. A system for optimizing a therapeutic regimen of docetaxel in combination of Granulocyte Colony-Stimulating Factor (G-CSF) or pegylated Granulocyte Colony-Stimulating Factor (G-CSF) for the treatment of cancer, the system comprising:
a docetaxel computer model for pharmacodynamics and pharmacokinetics of docetaxel based upon effects of docetaxel administration in vitro and/or in vivo to predict docetaxel-induced toxicity.

19. The system of claim 18, wherein the cancer is selected from the group comprising of breast cancer, lung cancer, prostate cancer, gastric cancer, head & neck cancer, melanoma, bladder cancer, neuroendocrine cancer, squamous carcinoma, cervical cancer, vulvar cancer, thyroid cancer, pancreatic cancer, renal cancer, esophageal cancer, rectal cancer, penile cancer, lymphoma, multiple myloma, Merkel cell tumors, ovarian cancer or colorectal cancer.

20. The system of claim 18, wherein the factors that may affect the pharmacokinetics of Docetaxel or Granulocyte Colony-Stimulating Factor (G-CSF) or pegylated Granulocyte Colony-Stimulating Factor (G-CSF), comprises: age, gender, alpha 1-acid glycoprotein, genetic variations of Cytochrome P450 genes, or CYP3A.

21. The system of claim 18, wherein the system is stored in a computer readable medium.

22. The system of claim 18, wherein the system operates over the interne.

23. The system of claim 18, wherein the system is stored in a hand-held calculator.

24. A method for treating cancer comprising administering a combination of docetaxel and supportive agent selected from Granulocyte Colony-Stimulating Factor (G-CSF) and pegylated Granulocyte Colony-Stimulating Factor (G-CSF) to a subject in need thereof, wherein docetaxel is administered in cycles of about 14 or 21 days and the supportive agent is administered 6 to 7 days following docetaxel administration, and
wherein when docetaxel is administered in cycles of about a week the supportive agent is administered 3 to 4 days following docetaxel administration.

25. A method of preventing chemotherapy-induced neutropenia comprising administering a chemotherapy agent and a supportive agent selected from Granulocyte Colony-Stimulating Factor (G-CSF) and pegylated Granulocyte Colony-Stimulating Factor(G-CSF) to a subject in need thereof, wherein the chemotherapy agent is administered in cycles of 14-21 days and the supportive agent is administered 6 to 7 days following administration of the chemotherapy agent, and
wherein when the chemotherapy agent is administered in cycles of about a week the supportive agent is administered 3 to 4 days following the administration of the chemotherapy agent.

26. The method of claim 25, wherein the chemotherapy-induced neutropenia is caused by chemotherapy agent comprising: alkylating agents, anti-metabolites, antitumour antibiotics, anthracyclines, plant alkaloids and terpenoids, taxanes, vinca alkaloid,s topoisomerase inhibitors, camptothecins or podophyllotoxins.

27. The method of claim 25, wherein the chemotherapy-induced neutropenia is caused by drugs comprising: docetaxel, doxorubicin, temozolomide, paclitaxel, irinotecan or carboplatin.

28. A method for treating cancer comprising administering a combination of chemotherapy agent and supportive agent selected from Granulocyte Colony-Stimulating Factor (G-CSF) and pegylated Granulocyte Colony-Stimulating Factor (G-CSF) to a subject in need thereof, wherein the supportive agent is administered in the range of two days before or after the day of the nadir of the plasma neutrophil count.

29. The method of claim 28, wherein the chemotherapy-induced neutropenia is caused by chemotherapy agent comprising: alkylating agents, anti-metabolites, antitumour antibiotics, anthracyclines, plant alkaloids and terpenoids, taxanes, vinca alkaloids, topoisomerase inhibitors, camptothecins or podophyllotoxins.

30. The method of claim 28, wherein the chemotherapy-induced neutropenia is caused by drugs comprising: docetaxel, doxorubicin, temozolomide, paclitaxel, irinotecan or carboplatin.

31. A method of determining an optimal therapeutic regimen forthe treatment of cancer with docetaxel comprising:
obtaining data to determine a docetaxel computer model for pharmacodynamics and pharmacokinetics of docetaxel based upon effects of docetaxel administration in vitro or in vivo;
obtaining data to determine a granulopoiesis computer model based upon measurement of neutrophils wherein the granulopoiesis computer model includes a process model for cells involved in neutrophil lineage;
creating a docetaxel/granulopoiesis computer model to predict an optimum treatment regimen for cancer by combination of the docetaxel computer model and granulopoiesis computer model;
performing in vitro or in vivo studies in which at least a single dose of docetaxel is administered and the in vitro or in vivo studies;
adjusting the docetaxel/granulopoiesis computer model based on comparison of results of the in vitro or in vivo studies and computer simulations using the docetaxel/granulopoiesis computer model;
determining one or more treatment schedules with docetaxel by the docetaxel/granulopoiesis model based upon results of computer simulations from the docetaxel/granulopoiesis computer model for docetaxel-induced toxicity; and
determining an optimal therapeutic regimen with docetaxel from the one or more treatment schedules by comparing results of computer simulations from the docetaxel/granulopoiesis computer model for reduced docetaxel-induced toxicity between the one or more treatment schedules.

32. The method of claim 31, wherein the cancer is selected from the group consisting of breast cancer, lung cancer, prostate cancer, gastric cancer, head and neck cancer, melanoma, bladder cancer, neuroendocrine cancer, squamous carcinoma, cervical cancer, vulvar cancer, thyroid cancer, pancreatic cancer, renal cancer, esophageal cancer, rectal cancer, penile cancer, lymphoma, multiple myloma, Merkel cell tumors, ovarian cancer, and colorectal cancer.

33. The method of claim 31, wherein the docetaxel-induced toxicity is neutropenia.

34. The method of claim 33, wherein the neutropenia is a grade 0, 1, 2, or 3/4 neutropenia.

35. The method of claim 31, wherein the granulopoeisis model accounts for effects of post-docetaxel administration of Granulocyte-Colony Stimulating Factor (G-CSF) or pegylated Granulocyte-Colony Stimulating Factor (G-CSF).

36. The method of claim 35, wherein G-CSF or pegylated G-CSF is administered at least 6-7 days post-docetaxel administration.

37. A method for determining optimal therapeutic regimen for the treatment of cancer with docetaxel in combination with Granulocyte Colony-Stimulating Factor (G-CSF) or pegylated Granulocyte Colony-Stimulating Factor (G-CSF) comprising:
obtaining data to determine a docetaxel computer model for pharmacodynamics and pharmacokinetics of docetaxel based upon effects of docetaxel administration in vitro or in vivo;
obtaining data to determine a granulopoiesis computer model based upon measurement of neutrophils wherein the granulopoiesis computer model includes a process model for cells involved in neutrophil lineage;
wherein the granulopoiesis computer model is adjusted based on comparison of results of in vitro or in vivo post-docetaxel administration of at least a single dose of G-CSF or pegylated G-CSF performed;
creating a docetaxel/granulopoiesis computer model to predict an optimum treatment regimen for cancer by combination of the docetaxel computer model and granulopoiesis computer model;
performing in vitro or in vivo studies in which at least a single dose of docetaxel is administered and the in vitro or in vivo studies;
adjusting the docetaxel/granulopoiesis model computer based on comparison of results of the in vitro or in vivo studies and computer simulations using the docetaxel/granulopoiesis computer model;
determining one or more treatment schedules with docetaxel and G-CSF or pegylated G-CSF using the docetaxel/granulopoiesis computer model based upon results of computer simulations from the docetaxel/granulopoiesis computer model for docetaxel-induced toxicity; and
determining the optimal therapeutic regimen from the one or more treatment schedules by comparing results of the computer simulations from the docetaxel/granulopoiesis computer model for reduced docetaxel-induced toxicity.

38. The method of claim 37, wherein the cancer is selected from the group comprising of breast cancer, lung cancer, prostate cancer, gastric cancer, head & neck cancer, melanoma, bladder cancer, neuroendocrine cancer, squamous carcinoma, cervical cancer, vulvar cancer, thyroid cancer, pancreatic cancer, renal cancer, esophageal cancer, rectal cancer, penile cancer, lymphoma, multiple myloma, Merkel cell tumors, ovarian cancer, and colorectal cancer.

39. The method of claim 37, wherein the docetaxel/granulopoiesis model is adjusted by at least one factor that may affect the pharmacokinetics of docetaxel, G-CSF, or pegylated G-CSF, comprising: age, gender, alpha 1-acid glycoprotein, genetic variations of Cytochrome P450 genes, or CYP3A.

40. The method of claim 37, wherein the docetaxel-induced toxicity is neutropenia.

41. The method of claim 40, wherein the neutropenia is a grade 0, 1, 2, or 3/4 neutropenia.

42. The method of claim 37, wherein G-CSF or pegylated G-CSF is administered at least 6-7 days post-docetaxel administration.

43. A system for optimizing a therapeutic regimen for the treatment of cancer with docetaxel, the system comprising:
a docetaxel computer model for pharmacodynamics and pharmacokinetics of docetaxel based upon effects of docetaxel administration in vitro or in vivo;
a granulopoiesis computer model that includes a process model for cells involved in neutrophil lineage;
a system model modifier wherein the system model modifier is adapted to modify the neutrophil lineage based on parameters specific to an individual to generate a modified system model;
wherein the parameters are determined from factors that may affect the pharmacokinetics of docetaxel schedule, G-CSF schedule, or pegylated G-CSF schedule;
wherein the system is operable to account for docetaxel-induced toxicity;
wherein the granulopoiesis model is adjusted based on comparison of results of post-docetaxel administration of at least a single dose of G-CSF or pegylated G-CSF in vitro or in vivo performed.

44. The system of claim 43, wherein the cancer is selected from the group comprising of breast cancer, lung cancer, prostate cancer, gastric cancer, head & neck cancer, melanoma, bladder cancer, neuroendocrine cancer, squamous carcinoma, cervical cancer, vulvar cancer, thyroid cancer, pancreatic cancer, renal cancer, esophageal cancer, rectal cancer, penile cancer, lymphoma, multiple myloma, Merkel cell tumors, ovarian cancer, and colorectal cancer.

45. The system of claim 43, wherein the system is stored in a computer readable medium.

46. The system of claim 43, wherein the system operates over the internet.

47. The system of claim 43, wherein the system is stored in a hand-held calculator.

48. A system for optimizing a therapeutic regimen of docetaxel in combination of Granulocyte Colony-Stimulating Factor (G-CSF) or pegylated Granulocyte Colony-Stimulating Factor (G-CSF) for the treatment of cancer, the system comprising:
a docetaxel computer model for pharmacodynamics and pharmacokinetics of docetaxel based upon effects of docetaxel administration in vitro or in vivo;
a granulopoiesis computer model based upon measurement of neutrophils wherein the granulopoiesis computer model includes a process model for cells involved in neutrophil lineage;
a system model modifier wherein the system model modifier is adapted to modify the neutrophil lineage based on parameters specific to an individual to generate a modified system model;
wherein the parameters are determined from factors that may affect the pharmacokinetics of docetaxel or G-CSF or pegylated G-CSF schedules;
wherein the system is operable to account for docetaxel-induced toxicity;
wherein the granulopoiesis computer model is adjusted based on comparison of results of in vitro or in vivo post-docetaxel administration of at least a single dose of G-CSF or pegylated G-CSF.

49. The system of claim 48, wherein the cancer is selected from the group comprising of breast cancer, lung cancer, prostate cancer, gastric cancer, head & neck cancer, melanoma, bladder cancer, neuroendocrine cancer, squamous carcinoma, cervical cancer, vulvar cancer, thyroid cancer, pancreatic cancer, renal cancer, esophageal cancer, rectal cancer, penile cancer, lymphoma, multiple myloma, Merkel cell tumors, ovarian cancer, and colorectal cancer.

50. The system of claim 48, wherein the factors that may affect the pharmacokinetics of Docetaxel or Granulocyte Colony-Stimulating Factor (G-CSF) or pegylated Granulocyte Colony-Stimulating Factor (G-CSF), comprises: age, gender, alpha 1-acid glycoprotein, genetic variations of Cytochrome P450 genes, or CYP3A.

51. The system of claim 48, wherein the system is stored in a computer readable medium.

52. The system of claim 48, wherein the system operates over the interne.

53. The system of claim 48, wherein the system is stored in a hand-held calculator.

54. A method for treating cancer comprising administering a combination of docetaxel and supportive agent selected from Granulocyte Colony-Stimulating Factor (G-CSF) and pegylated Granulocyte Colony-Stimulating Factor (G-CSF) to a subject in need thereof, wherein docetaxel is administered in cycles of about 14 or 21 days and the supportive agent is administered 6 to 7 days following docetaxel administration, and
wherein when docetaxel is administered in cycles of about a week the supportive agent is administered 3 to 4 days following docetaxel administration.

55. A method of preventing chemotherapy-induced neutropenia comprising administering a chemotherapy agent and a supportive agent selected from Granulocyte Colony-Stimulating Factor (G-CSF) and pegylated Granulocyte Colony-Stimulating Factor(G-CSF) to a subject in need thereof, wherein the chemotherapy agent is administered in cycles of 14-21 days and the supportive agent is administered 6 to 7 days following administration of the chemotherapy agent, and
wherein when the chemotherapy agent is administered in cycles of about a week the supportive agent is administered 3 to 4 days following the administration of the chemotherapy agent.

56. The method of claim 55, wherein the chemotherapy-induced neutropenia is caused by drugs comprising: docetaxel, doxorubicin, temozolomide, taxol, paclitaxel, irinotecan or carboplatin.
